(19) United States
(12) Patent Application Publication
(10) Pub. No.: US 2025/0194963 A1
(43) Pub. Date: Jun. 19, 2025

(54) STABILIZATION OF NAD(P)-DEPENDENT SENSOR WITH NEGATIVELY CHARGED MEMBRANE

(71) Applicant: Abbott Diabetes Care Inc., Alameda, CA (US)

(72) Inventors: Tianmei OUYANG, Saratoga, CA (US); Yagya R. OJHA, Alameda, CA (US); Lindsey PELSTER, Castro Valley, CA (US); Benjamin FELDMAN, Berkeley, CA (US); Jonathan D. MCCANLESS, Oakland, CA (US); Ahmed Hisham WALI, Alameda, CA (US)

(73) Assignee: Abbott Diabetes Care Inc., Alameda, CA (US)

(21) Appl. No.: 18/907,098

(22) Filed: Oct. 4, 2024

Related U.S. Application Data
(60) Provisional application No. 63/587,751, filed on Oct. 4, 2023.
(60) Provisional application No. 63/593,286, filed on Oct. 26, 2023.

(51) Int. Cl. A61B 5/145; A61B 5/1486

(52) U.S. Cl. CPC A61B 5/14546; A61B 5/14865

(57) ABSTRACT

The present disclosure provides analyte sensors comprising a sensing layer disposed upon a surface of a first working electrode, wherein the sensing layer comprises an NAD(P)-dependent enzyme and a supply of NAD(P); and a multilayered membrane that overcoats at least a part of the sensing layer and is permeable to an analyte, wherein the membrane comprises at least one layer of negatively charged polymer. The present disclosure also provides methods of using such analyte sensors for detecting one or more analytes preset in a biological sample and methods of manufacturing the analyte sensors.

### FIELD

The present disclosure provides analyte sensors comprising a sensing layer disposed upon a surface of a working electrode, wherein the sensing layer comprises an NAD(P)-dependent enzyme and a supply of NAD(P); and a multilayered membrane that overcoats at least a part of the sensing layer and is permeable to an analyte, wherein the membrane comprises at least one layer of negatively charged polymer, and wherein the negatively charged polymer limits transport of the supply of NAD(P) from the sensing layer. The present disclosure also provides methods of using such analyte sensors for detecting one or more analytes present in a biological sample and methods of manufacturing the analyte sensors.

### BACKGROUND

The detection of various analytes within an individual can sometimes be vital for monitoring the condition of their health, as deviations from normal analyte levels can be indicative of a physiological condition. For example, monitoring glucose levels can enable people suffering from diabetes to take appropriate corrective action including administering medicine or consuming a particular food or beverage products to avoid significant physiological harm. Other analytes can be desirable to monitor for other physiological conditions. In some instances, it can be desirable to monitor more than one analyte to monitor single or multiple physiological conditions, particularly if a person is suffering from comorbid conditions that result in simultaneous dysregulation of two or more analytes in combination with one another.

Analyte monitoring in an individual can take place periodically or continuously over a period of time. Periodic analyte monitoring can take place by withdrawing a sample of bodily fluid, such as blood or urine, at set time intervals and analyzing the same ex vivo. Periodic, ex vivo analyte monitoring can be sufficient to determine the physiological condition of many individuals. However, ex vivo analyte monitoring can be inconvenient or painful in some instances. Moreover, there is no way to recover lost data if an analyte measurement is not obtained at an appropriate time.

Continuous analyte monitoring can be conducted using one or more sensors that remain at least partially implanted within a tissue of an individual, such as dermally, subcutaneously or intravenously, so that analyses can be conducted in vivo. Implanted sensors can collect analyte data on-demand, at a set schedule, or continuously, depending on an individual's particular health needs and/or previously measured analyte levels. Analyte monitoring with an in vivo implanted sensor can be a more desirable approach for individuals having severe analyte dysregulation and/or rapidly fluctuating analyte levels, although it can also be beneficial for other individuals as well. Since implanted analyte sensors often remain within a tissue of an individual for an extended period of time, it can be highly desirable for such analyte sensors to be made from stable materials exhibiting a high degree of biocompatibility.

However, implantable sensors can be plagued by short life spans or reduced sensitivity. For example, many implantable sensors use enzymes for continuous monitoring of analyte levels in vivo and many of these enzymes rely on coenzymes for activity. For example, nicotinamide adenine dinucleotide (NAD) and nicotinamide adenine dinucleotide phosphate (NAD(P)) are two of the most important coenzymes found in living cells, and are frequently required for the activity of enzymes such as dehydrogenases that are found in implantable sensors. The amount of NAD or NAD(P) available for use by the enzymes present in implantable sensors can affect the sensitivity of the sensor to accurately monitor analyte levels in vivo. Under some circumstances, exogenous NAD or NAD(P) may not be present in sufficient quantities to support sensor operation or, even if sufficient exogenous quantities exists, such molecules are too large to readily diffuse to the area of the sensor that retains the enzyme dependent on NAD or NAD(P), which can lead to reduced sensitivity. Accordingly, there is a need in the art for sensors that control the amount of NAD or NAD(P) available and thus, allow the sensors to retain sensitivity.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

The present disclosure provides an analyte sensor comprising:
a proximal portion configured to be positioned above a user's skin; and
a distal portion configured to be transcutaneously positioned through the user's skin and in contact with the user's bodily fluid to detect or monitor the analyte in the bodily fluid in vivo; the distal portion comprising:
(i) a first sensing layer disposed upon a surface of a first working electrode, wherein the first sensing layer comprises an NAD(P)-dependent enzyme and a supply of NAD(P); and
(ii) a multilayered membrane that overcoats at least a part of the first sensing layer and is permeable to a first analyte, wherein the membrane comprises at least one layer of negatively charged polymer, and wherein the negatively charged polymer limits transport of the supply of NAD(P) from the first sensing layer.

In some embodiments, the multilayered membrane of the analyte sensor comprises from 1 to 3 layers of negatively charged polymer.

In some embodiments, the multilayered membrane of the analyte sensor comprises 1 layer of negatively charged polymer.

In some embodiments, the negatively charged polymer of the multilayered membrane comprises negatively charged sulfonate groups.

In some embodiments, the negatively charged polymer of the multilayered membrane comprises a copolymer of poly(tetrafluoroethylene) and a poly(perfluorosulfonic acid).

In some embodiments, the negatively charged polymer of the multilayered membrane comprises a copolymer of poly(tetrafluoroethylene) and a poly(perfluorosulfonic acid), wherein the perfluorosulfonic acid has repeat units represented by Formula (I):

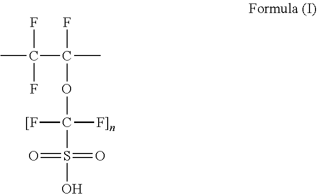

wherein n is an integer from 2 to 4.

In some embodiments, the negatively charged polymer of the multilayered membrane comprises AQUIVION® SO₃Li.

In some embodiments, the negatively charged polymer of the multilayered membrane comprises a sulfo-phenylated polyphenylene.

In some embodiments, the negatively charged polymer comprises a sulfo-phenylated polyphenylene polymer having repeat units represented by Formula (II):

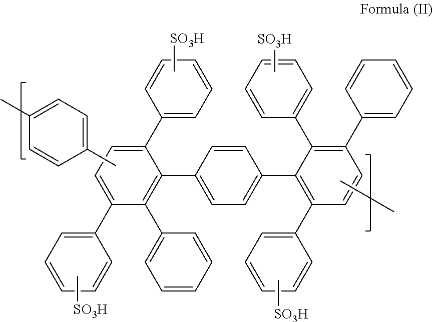

In some embodiments, the negatively charged polymer comprises PEMION®.

In some embodiments, the negatively charged polymer of the multilayered membrane comprises a copolymer of poly(vinylpyridine) and poly(styrene sulfonate).

In some embodiments, the negatively charged polymer of the multilayered membrane comprises poly(4-vinylpyridine-co-4-styrene sulfonic acid salt).

In some embodiments, the multilayered membrane of the analyte sensor further comprises at least one layer of a polymer selected from the group consisting of a polyvinylpyridine, a polyvinylimidazole, a polyacrylate, a polyurethane, a polyether urethane, a polystyrene, a polyacrylamide, and combinations thereof.

In some embodiments, the multilayered membrane of the analyte sensor comprises from 1 to 6 layers of a polymer that is not negatively charged.

In some embodiments, the multilayered membrane of the analyte sensor comprises from 1 to 3 layers of poly(4-vinylpyridine).

In some embodiments, the multilayered membrane of the analyte sensor comprises 2 layers of poly(4-vinylpyridine).

In some embodiments, the negatively charged polymer of the multilayered membrane limits transport of at least 80% of the supply of NAD(P) from the first sensing layer over 12 days.

In some embodiments, the analyte sensor exhibits less than a 20% decrease in signal over a period of 12 days.

In some embodiments, the NAD(P)-dependent enzyme in the first sensing layer is an NAD(P)-dependent dehydrogenase or a NAD(P)-dependent ketoreductase.

In some embodiments, the NAD(P)-dependent enzyme in the first sensing layer is a glucose dehydrogenase, a lactate dehydrogenase, an alcohol dehydrogenase, a p-hydroxybutyrate dehydrogenase, a phenylalanine dehydrogenase, an aldehyde reductase, or a ketoreductase.

In some embodiments, the NAD(P)-dependent enzyme in the first sensing layer is an aldehyde reductase or a ketoreductase.

In some embodiments, the analyte is selected from the group consisting of glucose, a ketone, an alcohol, lactate, and combinations thereof.

In some embodiments, the analyte is an alcohol.

In some embodiments, the first working electrode comprises carbon.

In some embodiments, the first sensing layer of the analyte sensor further comprises diaphorase.

In some embodiments, the first sensing layer of the analyte sensor further comprises a redox mediator.

In some embodiments, the analyte sensor further comprises: a second sensing layer disposed upon a surface of the first working electrode and responsive to a second analyte differing from the first analyte; wherein the second sensing layer comprises at least one enzyme responsive to the second analyte.

In some embodiments, the analyte sensor comprises:
(i) a first sensing layer disposed upon a surface of a first working electrode, wherein the first sensing layer comprises a NAD(P)-dependent enzyme and a supply of NAD(P); and
(ii) a multilayered membrane that overcoats at least a part of the first sensing layer and is permeable to a first analyte, wherein the membrane comprises at least one layer of negatively charged polymer, and wherein the negatively charged polymer limits transport of the supply of NAD(P) from the first sensing layer.
(iii) a second working electrode; and
(iv) a second sensing layer disposed upon a surface of the second working electrode and responsive to a second analyte differing from the first analyte;
wherein the second sensing layer comprises at least one enzyme responsive to the second analyte.

In some embodiments, the second sensing layer comprises an NAD(P)-dependent enzyme and a supply of NAD(P).

In some embodiments, the multilayered membrane overcoats at least a part of the second sensing layer.

The present disclosure also provides a method comprising:
(i) applying a potential to a first working electrode of an analyte sensor, wherein the analyte sensor comprises:
   (a) a first sensing layer disposed upon a surface of the first working electrode, wherein the first sensing layer comprises an NAD(P)-dependent enzyme and a supply of NAD(P); and
   (b) a multilayered membrane that overcoats at least a part of the first sensing layer and is permeable to a first analyte, wherein the membrane comprises at least one layer of negatively charged polymer, and wherein the negatively charged polymer limits transport of the supply of NAD(P) from the first sensing layer;
(ii) obtaining a first signal at or above an oxidation-reduction potential of the first sensing layer, the first signal being proportional to a concentration of a first analyte in a fluid contacting the first sensing layer; and
(iii) correlating the first signal to the concentration of the first analyte in the fluid.

### BRIEF DESCRIPTION OF THE DRAWINGS

The following figures are included to illustrate some aspects of the present disclosure and should not be viewed as exclusive embodiments. The subject matter disclosed is capable of considerable modifications, alterations, combinations, and equivalents in form and function, without departing from the scope of this disclosure.

FIG. 1 shows a diagram of an illustrative sensing system that can incorporate an analyte sensor of the present disclosure.

FIGS. 2A-2C show cross-sectional diagrams of analyte sensors including a single sensing layer.

FIGS. 3A-3C show cross-sectional diagrams of analyte sensors including two sensing layers.

FIG. 4 shows a cross-sectional diagram of an analyte sensor including two sensing layers.

FIGS. 5A-5C show perspective views of analyte sensors including two sensing layers upon separate working electrodes.

FIG. 6 shows a diagram of an alcohol enzyme system that can be used for detecting alcohol according to the present disclosure.

FIG. 7 is a diagram of an analyte sensor comprising a screen-printed carbon electrode, a sensing layer disposed on the carbon electrode, wherein the sensing layer comprises an NAD(P)-dependent enzyme and a supply of NAD(P); and a multilayered membrane that overcoats the sensing layer, wherein the multilayered membrane comprises a negatively charged polymer.

FIG. 8 is a line graph showing sensor current (nA) versus time (hours) of an exemplary analyte sensor of the present disclosure comprising a poly(4-vinylpyridine) (PVP) membrane (bottom line) as a control compared to an analyte sensor comprising a multilayered membrane comprising a layer of poly(4-vinylpyridine), a layer of AQUIVION® (Aqv-Li), a second layer of poly(4-vinylpyridine), and a layer of osmium-containing poly(4-vinylpyridine)-based polymer (top line).

FIG. 9 is a line graph showing sensor current (nA) versus time (hours) of an exemplary analyte sensor of the present disclosure comprising a poly(4-vinylpyridine) membrane (bottom line) as a control compared to an analyte sensor comprising a multilayered membrane comprising a layer of poly(4-vinylpyridine), a layer of poly(4-vinylpyridine-co-4-styrene sulfonic acid salt), a second layer of poly(4-vinylpyridine), and a layer of osmium-containing poly(4-vinylpyridine)-based polymer (top line).

FIG. 10 is a line graph showing sensor current (nA) versus time (seconds) of an exemplary analyte sensor of the present disclosure comprising a multilayered membrane comprising a layer of poly(4-vinylpyridine), a layer of PEMION®, a second layer of poly(4-vinylpyridine), and a layer of osmium-containing poly(4-vinylpyridine)-based polymer.

### DETAILED DESCRIPTION

The present disclosure is directed to analyte sensors comprising one or more sensing layers that include an NAD(P)-dependent enzyme. In particular, analyte sensors of the present disclosure include a supply of the cofactor NAD(P) for the NAD(P)-dependent enzyme in a sensing layer.

The use of a multilayered membrane comprising a negatively charged polymer within an analyte sensor can overcome some of the limitations associated with analyte sensors that include an NAD(P)-dependent enzyme. For example, the amount of exogenous NAD(P) present in the environment surrounding the sensor might not be in sufficient quantities to support analyte sensor operation, which can result in reduced sensitivity of the sensor. In addition, even if sufficient exogenous NAD(P) exists in the environment surrounding the analyte sensor, the molecular size of NAD(P) can prevent the molecule from diffusing through the surrounding sensor membrane to reach the one or more NAD(P)-dependent enzymes present in the sensing layer of the analyte sensor.

The present disclosure provides analyte sensors that include a multilayered membrane comprising a negatively charged polymer that allows the supply of NAD(P) to remain constant in the sensing layer permitting the sensor to retain sensitivity. In some embodiments, the sensing layer includes an NAD(P) supply that is overcoated with a multilayered membrane comprising a negatively charged polymer that controls diffusion of NAD(P) from the sensing layer to maintain a sufficient concentration of NAD(P) for the sensing layer during use of the analyte sensor.

The present disclosure further provides methods of detecting an analyte using the disclosed sensors and methods of manufacturing the disclosed analyte sensors.

### I. Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the term “about” means±10% of the specified value, unless otherwise indicated.

The term “at least” prior to a number or series of numbers is understood to include the number associated with the term “at least,” and all subsequent numbers or integers that could logically be included, as clear from context. When “at least” is present before a series of numbers or a range, it is understood that “at least” can modify each of the numbers in the series or range. For example, “at least 3” means at least 3, at least 4, at least 5, etc. When “at least” is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the terms “measure,” “measuring,” and “measured” can encompass the meaning of a respective one or more of the terms “determine,” “determining,” “determined,” “calculate,” “calculating,” and “calculated.”

As used herein, an “analyte” is a substrate that is subject to be measured or detected. The analyte can be from, for example, a biofluid and can be tested in vivo, ex vivo, or in vitro.

As used herein, a “sensor” is a device configured to detect the presence and/or measure the level of an analyte in a sample via electrochemical oxidation and reduction reactions on the sensor. These reactions are transduced to an electrical signal that can be be indicative of presence and be correlated to an amount, concentration, or level of an analyte in the sample.

As used herein, a “working electrode” is an electrode at which the analyte (or additional compound(s) whose level depends on the level of the analyte) is electrooxidized or electroreduced with or without the agency of an electron transfer agent.

As used herein, a “counter electrode” refers to an electrode paired with the working electrode, through which passes a current equal in magnitude and opposite in sign to the current passing through the working electrode. In the context of embodiments of the present disclosure, the term “counter electrode” includes both a) counter electrodes and b) counter electrodes that also function as reference electrodes (i.e., counter/reference electrodes), unless otherwise indicated.

As used herein, a “reference electrode” includes both a) reference electrodes and b) reference electrodes that also function as counter electrodes (i.e., counter/reference electrodes), unless otherwise indicated.

As used herein, “electrolysis” is the electrooxidation or electroreduction of a compound either directly at an electrode or via one or more electron transfer agents.

As used herein, an “electron transfer agent” is a compound that carries electrons between the analyte and the working electrode, either directly, or in cooperation with other electron transfer agents. One example of an electron transfer agent is a redox mediator.

As used herein, a “redox mediator” is an electron-transfer agent for carrying electrons between an analyte, an analyte-reduced or analyte-oxidized, enzyme, and an electrode, either directly, or via one or more additional electron-transfer agents. A redox mediator that includes a polymeric backbone can also be referred to as a “redox polymer.”

A “reactive group” is a functional group of a molecule (e.g., a polymer, a crosslinking agent, an enzyme) that is capable of reacting with another compound to couple at least a portion (e.g., another reactive group) of that other compound to the molecule. Reactive groups include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

As used herein, a “sensing layer” is a component of the sensor including constituents that facilitate the electrolysis of the analyte. The sensing layer can include constituents such as a redox mediator (e.g., an electron transfer agent or a redox polymer), a catalyst (e.g., an analyte-specific reactant), which catalyzes a reaction of the analyte to produce a response at the working electrode, or both an electron transfer agent and a catalyst. In some embodiments of the present disclosure, a sensor includes a sensing layer that is non-leachably disposed in proximity to or on the working electrode.

As used herein, a “sensing element” is an application or region of an analyte-specific reactant disposed with the sensing layer. As such, a sensing element is capable of interacting with the analyte. A sensing layer can have more than one sensing element making up the analyte detection area disposed on the working electrode. In some embodiments, the sensing element includes an analyte-specific reactant and an electron transfer agent (e.g., electron transfer agent). In some embodiments, the sensing element includes an analyte-specific reactant, a redox mediator, and a crosslinker.

As used herein, “crosslinking agent” or “crosslinker” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinkings at the same time.

A “membrane solution” is a solution that contains the components for crosslinking and forming the membrane, including, e.g., polymer (e.g., a modified polymer containing heterocyclic nitrogen groups), a crosslinking agent, and a solvent (e.g., a buffer or an alcohol-buffer mixed solvent).

As used herein, a “biofluid” is any bodily fluid or bodily fluid derivative in which the analyte can be measured. Examples of biofluid include, for example, dermal fluid, subcutaneous fluid, interstitial fluid, plasma, blood (e.g., from a vein or blood vessel), lymph, synovial fluid, cerebrospinal fluid, saliva, bronchoalveolar lavage, amniotic fluid, sweat, or tears.

The term “patient” refers to a living animal, and thus encompasses a living mammal and a living human, for example. The term “user” can be used herein as a term that encompasses the term “patient.”

All ranges disclosed herein are inclusive of the endpoints, and the endpoints are independently combinable with each other. Each range disclosed herein constitutes a disclosure of any point or sub-range lying within the disclosed range. For example, the range from X to Y, is inclusive of X and Y. And, the range between X and Y, is inclusive of X and Y.

The phrase “enzyme composition” refers to a composition that includes one or more enzymes for detecting and/or measuring an analyte. In some non-limiting embodiments, the enzyme compositions can include one or more enzymes, polymers, redox mediators, and/or crosslinkers.

As used herein, the phrase “multi-component membrane” refers to a membrane comprising two or more types of membrane polymers.

As used herein, the phrase “multilayered membrane” refers to a membrane system comprising two of more layers of membrane polymer. The two or more layers of membrane polymer can comprise multiple layers of the same membrane polymer as long as there is at least one different membrane polymer layer between the two membrane polymer layers comprising the same membrane polymer.

As used herein, the term “NAD(P)” refers to the cofactor NAD (and its reduced form NADH) and/or NADP (and its reduced form NADPH).

As used herein, the term “NAD(P)-dependent enzyme” refers to an enzyme that uses NAD (and its reduced form NADH) and/or NADP (and its reduced form NADPH) as a coenzyme in a redox reaction.

### II. Analyte Sensors

### Sensors, Compositions, and Methods of the Disclosure

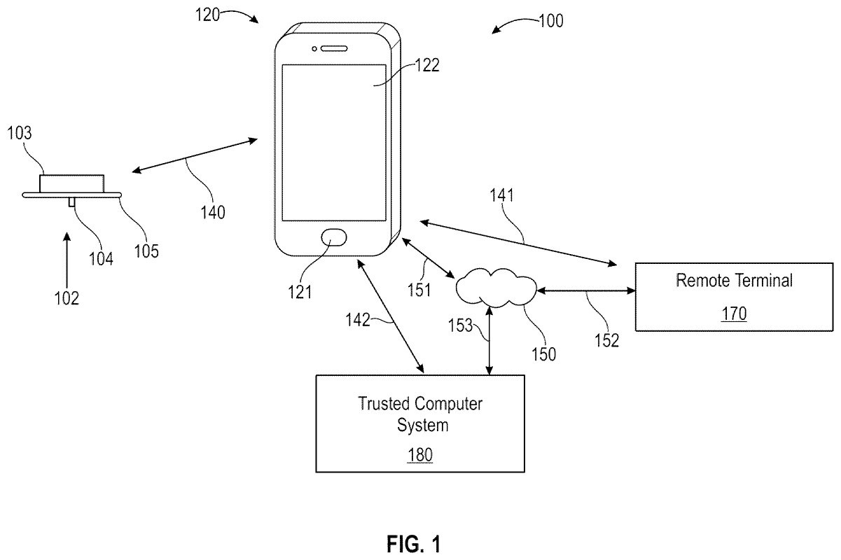

Before describing the analyte sensors of the present disclosure and their components in further detail, a brief overview of suitable in vivo analyte sensor configurations and sensor systems employing the analyte sensors will be provided so that the embodiments of the present disclosure can be better understood. FIG. 1 shows a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to some embodiments. Reader device 120 can be a multi-purpose smartphone or a dedicated electronic reader instrument. While only one reader device 120 is shown, multiple reader devices 120 can be present in some instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to some embodiments, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to some embodiments, by individuals other than a primary user who have an interest in the user's analyte levels. Reader device 120 can include display 122 and optional input component 121. Display 122 can include a touch-screen interface, according to some embodiments.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to some embodiments.

Sensor 104 is adapted to be at least partially inserted into a tissue of interest, such as within the dermal or subcutaneous layer of the skin where sensor 104 can comprise a proximal portion and a distal portion. In some embodiments, for example, the distal portion of the sensor is configured for in vivo placement, e.g., for transcutaneous positioning through the skin of a subject. According to some embodiments, an introducer (e.g., a needle or a sharp) can create an insertion path through the subject's skin during the transcutaneous positioning of the distal portion of the sensor. In some embodiments, the sensor can comprise a member capable of penetrating the skin of a subject. In such embodiments, for example, the member can comprise an insertable tip, tail, probe, or needle capable of penetrating the skin of a subject. According to some embodiments, the distal portion of sensor 104 comprises an implantable portion of sufficient length for insertion to a desired depth in a given tissue. The implantable portion can include at least one working electrode. In some configurations, the implantable portion can include a sensing layer for detecting an analyte. A counter electrode can be present in combination with the at least one working electrode. Particular electrode configurations upon the implantable portion are described in more detail below. According to another aspect of the embodiments, the proximal portion of the sensor can be configured to remain above the skin (ex vivo), and can be configured to be electrically coupled with the circuitry disposed in the sensor housing 103 of sensor control device 102.

The sensing layer can be configured for detecting a particular analyte. For example, but not by way of limitation, the disclosed analyte sensors include at least one sensing layer configured to detect an analyte (e.g., glucose, ketone). In some embodiments, a sensor of the present disclosure includes two sensing layers, where each sensing layer is configured to detect a different analyte. Alternatively, the two sensing layers can be configured to detect the same analyte. In some embodiments, a first sensing layer can be configured to detect an analyte (e.g., glucose) and a second sensing layer can be configured to detect the first (i.e., same) analyte or a second analyte different from the first analyte (e.g., ketone, creatinine).

In some embodiments of the present disclosure, one or more analytes can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In some embodiments, analyte sensors of the present disclosure can be adapted for assaying dermal fluid or interstitial fluid to determine a concentration of one or more analytes in vivo. In some embodiments, the biological fluid is interstitial fluid.

Referring still to FIG. 1, sensor 104 can automatically forward data to reader device 120. For example but not by the way of limitation, analyte concentration data (i.e., glucose concentration) can be communicated automatically and periodically, such as at a some frequency as data is obtained or after a some time period has passed, with the data being stored in a memory until transmittal (e.g., every minute, five minutes, or other predetermined time period). In some embodiments, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In some embodiments, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In some illustrative embodiments, the introducer can include a needle or similar sharp. As would be readily recognized by a person skilled in the art, other types of introducers, such as sheaths or blades, can be present in alternative embodiments. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more embodiments. After opening the access pathway, the needle or other introducer can be withdrawn so that it does not represent a sharps hazard. In some embodiments, suitable needles can be solid or hollow, beveled or non-beveled, and/or circular or non-circular in cross-section. In some embodiments, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In some embodiments, a tip of the needle (while present) can be angled over the terminus of sensor 104, such that the needle penetrates a tissue first and opens an access pathway for sensor 104. In some embodiments, sensor 104 can reside within a lumen or groove of the needle, with the needle similarly opening an access pathway for sensor 104. In either case, the needle is subsequently withdrawn after facilitating sensor insertion.

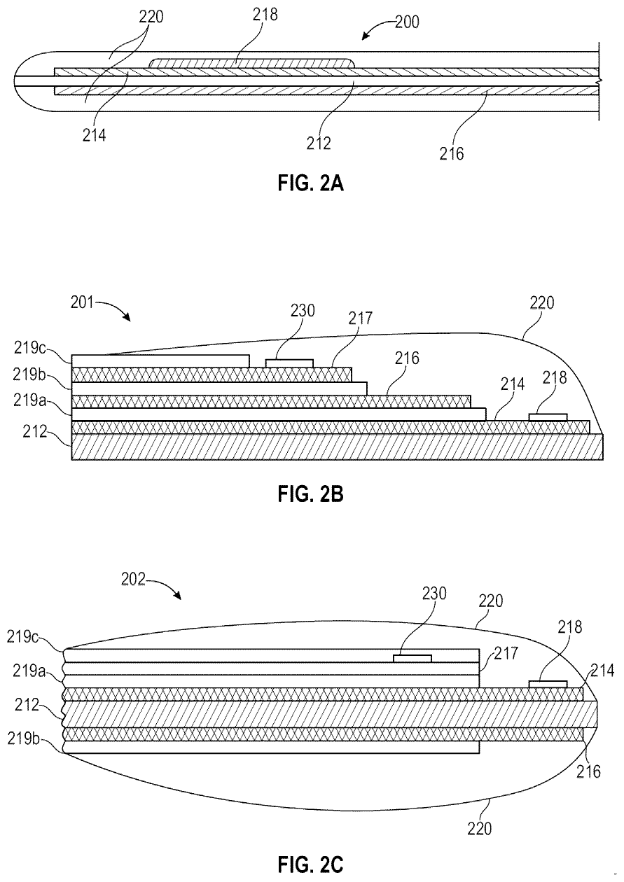

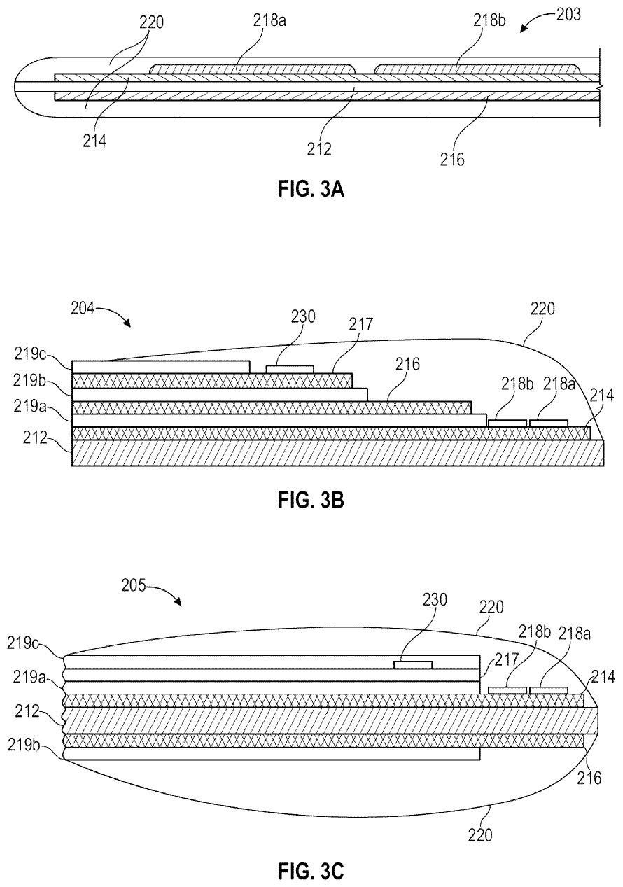

Sensor configurations featuring a single sensing layer that is configured for the detection of a corresponding single analyte can employ two-electrode or three-electrode detection motifs, as described further herein in reference to FIGS. 2A-2C. Sensor configurations featuring two different sensing layers for detection of separate analytes, either upon separate working electrodes or upon the same working electrode, are described separately thereafter in reference to FIGS. 3A-5C. Sensor configurations having multiple working electrodes can be particularly advantageous for incorporating two different sensing layers within the same implantable portion, since the signal contribution from each sensing layer can be determined more readily.

When a single working electrode is present in an analyte sensor, three-electrode sensor configurations can include a working electrode, a counter electrode, and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the implantable portion. Suitable sensor configurations can be substantially flat in shape, substantially cylindrical in shape or any other suitable shape. In any of the sensor configurations disclosed herein, the various electrodes can be electrically isolated from one another by a dielectric material or similar insulator.

Analyte sensors featuring multiple working electrodes can similarly include at least one additional electrode. When one additional electrode is present, the one additional electrode can function as a counter/reference electrode for each of the multiple working electrodes. When two additional electrodes are present, one of the additional electrodes can function as a counter electrode for each of the multiple working electrodes and the other of the additional electrodes can function as a reference electrode for each of the multiple working electrodes.

FIG. 2A shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Sensing layer 218 is disposed as at least one layer upon at least a portion of working electrode 214. Sensing layer 218 can include multiple spots or a single spot configured for detection of an analyte, as discussed further herein.

Referring still to FIG. 2A, membrane 220 overcoats at least sensing layer 218. In some embodiments, membrane 220 can also overcoat some or all of working electrode 214 and/or counter/reference electrode 216, or the entirety of analyte sensor 200. One or both faces of analyte sensor 200 can be overcoated with membrane 220. Membrane 220 can include one or more polymeric membrane materials having capabilities of limiting analyte flux to sensing layer 218 (i.e., membrane 220 is a mass transport limiting membrane having some permeability for the analyte of interest). In some embodiments, and further described below, membrane 220 is not crosslinked. Analyte sensor 200 can be operable for assaying an analyte by any of coulometric, amperometric, voltammetric, or potentiometric electrochemical detection techniques.

FIGS. 2B and 2C show diagrams of illustrative three-electrode analyte sensor configurations, which are also compatible for use in the disclosure herein. Three-electrode analyte sensor configurations can be similar to that shown for analyte sensor 200 in FIG. 2A, except for the inclusion of additional electrode 217 in analyte sensors 201 and 202 (FIGS. 2B and 2C). With additional electrode 217, counter/reference electrode 216 can then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, and not by the way of limitation, as depicted in FIG. 2B, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 2C. Thus, in certain embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to that depicted in FIGS. 2B and 2C. As with sensor 200 shown in FIG. 2A, sensing layer 218 in analyte sensors 201 and 202 can include multiple spots or a single spot. Additionally, analyte sensors 201 and 202 can be operable for assaying an analyte by any of coulometric, amperometric, voltammetric, or potentiometric electrochemical detection techniques.

Like analyte sensor 200, membrane 220 can also overcoat sensing layer 218, as well as other sensor components, in analyte sensors 201 and 202, thereby serving as a mass transport limiting membrane. In some embodiments, the additional electrode 217 can be overcoated with membrane 220. Although FIGS. 2B and 2C have depicted electrodes 214, 216, and 217 as being overcoated with membrane 220, it is to be recognized that in some embodiments only working electrode 214 is overcoated. Moreover, the thickness of membrane 220 at each of electrodes 214, 216, and 217 can be the same or different. As in two-electrode analyte sensor configurations (FIG. 2A), one or both faces of analyte sensors 201 and 202 can be overcoated with membrane 220 in the sensor configurations of FIGS. 2B and 2C, or the entirety of analyte sensors 201 and 202 can be overcoated. Accordingly, the three-electrode sensor configurations shown in FIGS. 2B and 2C should be understood as being non-limiting of the embodiments disclosed herein, with alternative electrode and/or layer configurations remaining within the scope of the present disclosure.

FIG. 3A shows an illustrative configuration for sensor 203 having a single working electrode with two different sensing layers disposed thereon. FIG. 3A is similar to FIG. 2A, except for the presence of two sensing layers upon working electrode 214: first sensing layer 218a and second sensing layer 218b, which are responsive to different analytes and are laterally spaced apart from one another upon the surface of working electrode 214. Sensing layers 218a and 218b can include multiple spots or a single spot configured for detection of each analyte. The composition of membrane 220 can vary or be compositionally the same at sensing layers 218a and 218b. First sensing layer 218a and second sensing layer 218b can be configured to detect their corresponding analytes at working electrode potentials that differ from one another, as discussed further below.

FIGS. 3B and 3C show cross-sectional diagrams of illustrative three-electrode sensor configurations for sensors 204 and 205, respectively, each featuring a single working electrode having first sensing layer 218a and second sensing layer 218b disposed thereon. FIGS. 3B and 3C are otherwise similar to FIGS. 2B and 2C and can be better understood by reference thereto. As with FIG. 3A, the composition of membrane 220 can vary or be compositionally the same at sensing layers 218a and 218b.

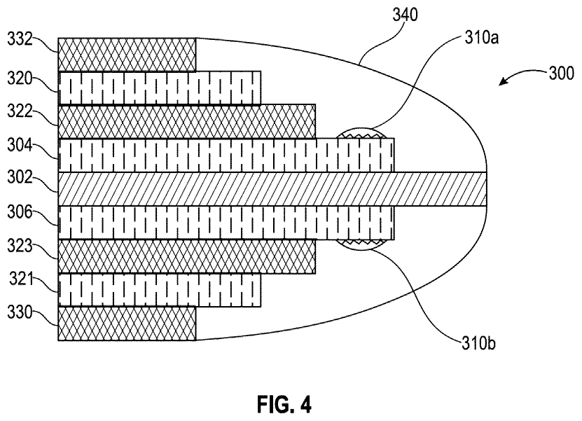

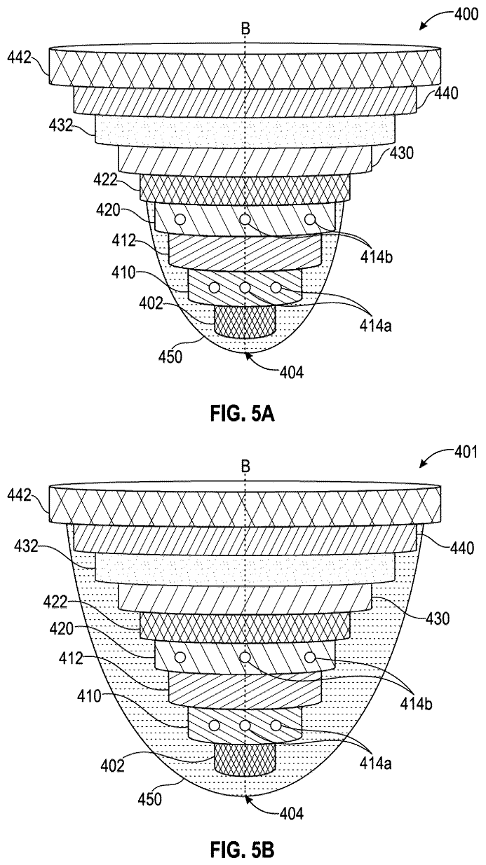

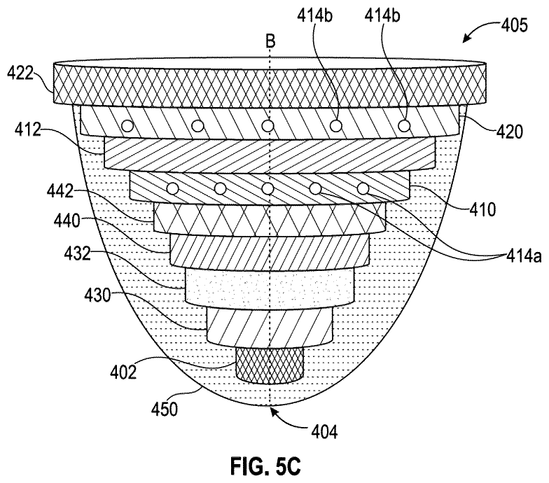

Illustrative sensor configurations having multiple working electrodes, specifically two working electrodes, are described in further detail in reference to FIGS. 4-5C. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte, e.g., for the detection of a third and/or fourth analyte.

FIG. 4 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First sensing layer 310a is disposed upon the surface of working electrode 304, and second sensing layer 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least sensing layers 310a and 310b, according to various embodiments, with other components of analyte sensor 300 or the entirety of analyte sensor 300 optionally being overcoated with membrane 340.

Like analyte sensors 200, 201, and 202, analyte sensor 300 can be operable for assaying an analyte by any of coulometric, amperometric, voltammetric, or potentiometric electrochemical detection techniques.

Alternative sensor configurations having multiple working electrodes and differing from the configuration shown in FIG. 4 can feature a counter/reference electrode instead of separate counter and reference electrodes 320, 321, and/or feature layer and/or membrane arrangements varying from those expressly depicted. For example, and not by the way of limitation the positioning of counter electrode 320 and reference electrode 321 can be reversed from that depicted in FIG. 4. In addition, working electrodes 304 and 306 need not necessarily reside upon opposing faces of substrate 302 in the manner shown in FIG. 4.

Although suitable sensor configurations can feature electrodes that are substantially planar in character, it is to be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described herein below. FIGS. 5A-5C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 5A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 5A, first sensing layers 414a and second sensing layers 414b, which are responsive to different analytes or the same analyte, are disposed upon the exposed surfaces of working electrodes 410 and 420, respectively, thereby allowing contact with a fluid to take place for sensing. Although sensing layers 414a and 414b have been depicted as three discrete spots in FIG. 5A, it is to be appreciated that fewer or greater than three spots, including a continuous layer of sensing layer, can be present in alternative sensor configurations.

In FIG. 5A, sensor 400 is partially coated with membrane 450 upon working electrodes 410 and 420 and sensing layers 414a and 414b disposed thereon. FIG. 5B shows an alternative sensor configuration in which the substantial entirety of sensor 401 is overcoated with membrane 450. Membrane 450 can be the same or vary compositionally at sensing layers 414a and 414b.

It is to be further appreciated that the positioning of the various electrodes in FIGS. 5A and 5B can differ from that expressly depicted. For example, the positions of counter electrode 430 and reference electrode 440 can be reversed from the depicted configurations in FIGS. 5A and 5B. Similarly, the positions of working electrodes 410 and 420 are not limited to those that are expressly depicted in FIGS. 5A and 5B. FIG. 5C shows an alternative sensor configuration to that shown in FIG. 5B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of sensing layers 414a and 414b (five discrete sensing spots illustratively shown in FIG. 5C), thereby facilitating an increased signal strength in some cases. Similarly, central substrate 402 can be omitted in any concentric sensor configuration disclosed herein, wherein the innermost electrode can instead support subsequently deposited layers.

Several parts of the sensor are further described below.

### III. General Structure of the Analyte Sensor System

In some embodiments, the present disclosure is directed to a analyte sensor comprising:
a proximal portion configured to be positioned above a user's skin; and
a distal portion configured to be transcutaneously positioned through the user's skin and in contact with the user's bodily fluid to detect or monitor the analyte in the bodily fluid in vivo; the distal portion comprising:
(i) a first sensing layer disposed upon a surface of a first working electrode, wherein the first sensing layer comprises an NAD(P)-dependent enzyme and a supply of NAD(P); and
(ii) a multilayered membrane that overcoats at least a part of the first sensing layer and is permeable to a first analyte, wherein the membrane comprises at least one layer of negatively charged polymer, and wherein the negatively charged polymer limits transport of the supply of NAD(P) from the first sensing layer.

In some embodiments, the present disclosure is directed to a analyte sensor comprising:
a proximal portion configured to be positioned above a user's skin; and
a distal portion configured to be transcutaneously positioned through the user's skin and in contact with the user's bodily fluid to detect or monitor the analyte in the bodily fluid in vivo; the distal portion comprising:
(i) a first sensing layer disposed upon a surface of a first working electrode, wherein the first sensing layer comprises an NAD(P)-dependent enzyme and a supply of NAD(P);
(ii) a multilayered membrane that overcoats at least a part of the first sensing layer and is permeable to a first analyte, wherein the membrane comprises at least one layer of negatively charged polymer, and wherein the negatively charged polymer limits transport of the supply of NAD(P) from the first sensing layer;
(iii) a second working electrode; and
(iv) a second sensing layer disposed upon a surface of the second working electrode and responsive to a second analyte differing from the first analyte;
wherein the second sensing layer comprises at least one enzyme responsive to the second analyte.

It was surprisingly discovered that by replacing a traditional poly(4-vinylpyridine)-based membrane with a multilayered membrane that comprises at least one layer of negatively charged polymer, that the negatively charged polymer prevents NAD(P) from leaking out of the sensing layer. Since NAD(P) is negatively charged, overcoating a sensing layer with a multilayered membrane comprising a negatively charged polymer retains NAD(P) in the sensing layer and prevents its diffusion through the membrane.

### 1. Working Electrode

In the analyte sensor, the working electrode can be any suitable conductive material, such as carbon, gold, palladium, or platinum. The sensing layer senses a desired analyte (e.g., glucose) and can be continuously or discontinuously disposed on at least a portion of the working electrode. A discontinuous application means that the sensing layer forms a discrete shape on the working electrode, such as a spot, a line, or a plurality (i.e., an array) of spots and/or lines. The number of spots or lines is not considered to be particularly limited, but can range from about 2 to about 10, from about 3 to about 8, or from about 4 to about 6. In some embodiments, the number of spots or lines can be 2, 3, 4, 5, 6, 7, 8, 9, or 10. In some embodiments, the sensing layer can be continuous on the working electrode. In some embodiments, the sensing layer can be discontinuous on the working electrode.

In some embodiments, the working electrode is a screen-printed carbon electrode.

### 2. Sensing Layer

In some embodiments, the working electrode can comprise at least one sensing layer. In some embodiments, the working electrode can comprise one sensing layer. In some embodiments, the working electrode can comprise two sensing layers. In some embodiments, the working electrode can comprise a first sensing layer and a second sensing layer, wherein the analyte for the first sensing layer is different from the analyte for the second sensing layer. In this instance, the first sensing layer and second sensing layer can form an array of multiple spots of each sensing layer, in which some spots sense a first analyte (e.g., glucose) and other spots sense a second analyte different from the first analyte (e.g., ketone, creatinine). Each spot can range in size from about 0.01 to 1 mm² in diameter.

The total size of the sensing layer or areas (combined area of all spots, layers, or sensing layers) can be from about 0.05 mm² to about 100 mm². In some embodiments, the total size can be about 100 mm² or less, about 75 mm² or less, about 50 mm² or less, about 40 mm² or less, about 30 mm² or less, about 25 mm² or less, about 15 mm² or less, about 10 mm² or less, about 5 mm² or less, about 1 mm² or less, or about 0.1 mm² or less. In some embodiments, the total size of the sensing layer or areas ranges from about 0.05 to about 0.1 mm², from about 0.05 to about 100 mm², from about 0.1 to about 50 mm², from about 0.5 to about 30 mm², from about 1 to about 20 mm², or from about 1 to about 15 mm².

The sensing layer or areas can typically have a thickness that ranges from about 0.1 μm to about 10 μm. For example, each sensing layer can be 0.1 μm thick or more (e.g., 0.2 μm or more, 0.3 μm or more, 0.5 μm or more, 0.8 μm or more, 1 μm or more, 2 μm or more, 3 μm or more, 5 μm or more, or 8 μm or more) and typically will have a thickness of 10 μm or less (e.g., 8 μm or less, 5 μm or less, 3 μm or less, 2 μm or less, 1 μm or less, 0.8 μm or less, 0.5 μm or less, 0.3 μm or less, or 0.2 μm or less). In some embodiments, each sensing layer has a thickness from about 0.1 μm to about 10 μm, from about 0.2 μm to about 8 μm, from about 0.5 μm to about 5 μm, from about 1 μm to about 4 μm, or from about 1 μm about 2 μm.

In some embodiments, a conductive material such as, for example, carbon nanotubes, graphene, or metal nanoparticles, can be combined within the sensing layer or layers to promote rapid attainment of a steady state current. Conductive material can be included in a range from about 0.1% to about 50% by weight (pbw) of the sensing layer (e.g., from about 1 pbw to about 50 pbw, from about 1 pbw to about 10 pbw, or from about 0.1 pbw to about 10 pbw).

### 3. Cofactor Supply

The present disclosure provides analyte sensors that include a supply of a cofactor in the sensing layer. For example, but not by way of limitation, the present disclosure provides analyte sensors that include a supply of a cofactor that allows the controlled release of the cofactor over an extended period of the time.

In some embodiments, the sensing layer that comprises a supply of cofactor can be coated with a multilayered membrane comprising at least one negatively charged polymer that controls diffusion of the cofactor from the sensing layer to maintain a sufficient concentration of the cofactor in the sensing layer during use of the analyte sensor. The exact amount of the cofactor supply present within an analyte sensor can vary based on the particular application of the analyte sensor, e.g., which analyte is being detected, the duration of analyte detection, and the conditions under which the detection of the analyte occurs.

In some embodiments, the cofactor is NAD(P). Non-limiting examples of NAD(P) derivatives are disclosed in WO 2007/012494 and WO 1998/033936, the contents of each which are incorporated herein by reference in their entireties. In some embodiments, the present disclosure provides analyte sensors that can include a supply of NAD(P) in the sensing layer that allows the controlled release of NAD(P) or derivative thereof over an extended period of the time. In some embodiments, the sensing layer comprising a supply of NAD(P) can be coated with a multilayered membrane comprising at least one negatively charged polymer that controls diffusion of NAD(P) from the NAD(P) supply to maintain a sufficient concentration of NAD(P) in a sensing layer, comprising one or more NAD(P)-dependent enzymes during use of the analyte sensor.

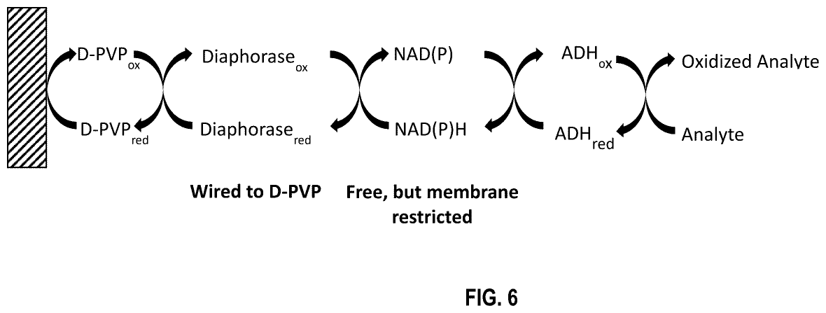

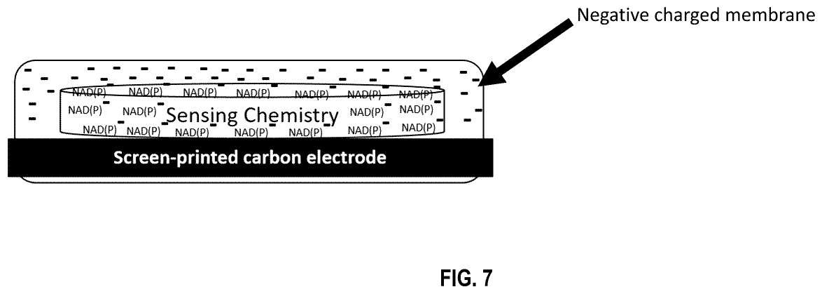

A non-limiting embodiment of an analyte sensor that includes a sensing layer with a supply of NAD(P) is provided in FIG. 7. For example, but not by way of limitation, the supply of NAD(P) can be deposited onto a substrate, e.g., a carbon electrode, as shown in FIG. 6.

In some embodiments, the amount of NAD(P) present within a sensing layer can vary depending on the duration of use of the analyte sensor. In some embodiments, NAD(P) can be present in a sensing layer in an amount from about 0.1 μg to about 1,000 μg. In some embodiments, from about 0.1 μg to about 900 μg, from about 0.1 μg to about 800 μg, from about 0.1 μg to about 700 μg, from about 0.1 μg to about 600 μg, from about 0.1 μg to about 500 μg, from about 0.1 μg to about 400 μg, from about 0.1 μg to about 300 μg, from about 0.1 μg to about 200 μg, from about 0.1 μg to about 100 μg, from about 0.1 μg to about 90 μg, from about 0.1 μg to about 80 μg, from about 0.1 μg to about 70 μg, from about 0.1 μg to about 60 μg, from about 0.1 μg to about 50 μg, from about 0.1 μg to about 40 μg, from about 0.1 μg to about 30 μg, from about 0.1 μg to about 20 μg, from about 0.1 μg to about 10 μg, from about 0.1 μg to about 9 μg, from about 0.1 μg to about 8 μg, from about 0.1 μg to about 7 μg, from about 0.1 μg to about 6 μg, from about 0.1 μg to about 5 μg, from about 0.1 μg to about 4 μg, from about 0.1 μg to about 3 μg, from about 0.1 μg to about 2 μg, from about 0.1 μg to about 1 μg, from about 0.1 μg to about 0.9 μg, from about 0.1 μg to about 0.8 μg, from about 0.1 μg to about 0.7 μg, from about 0.1 μg to about 0.6 μg, from about 0.1 μg to about 0.5 μg, from about 0.1 μg to about 0.4 μg, from about 0.1 μg to about 0.3 μg, from about 0.1 μg to about 0.2 μg, from about 0.2 μg to about 1,000 μg, from about 0.3 μg to about 1,000 μg, from about 0.4 μg to about 1,000 μg, from about 0.5 μg to about 1,000 μg, from about 0.6 μg to about 1,000 μg, from about 0.7 μg to about 1,000 μg, from about 0.8 μg to about 1,000 μg, from about 0.9 μg to about 1,000 μg, from about 1 μg to about 1,000 μg, from about 2 μg to about 1,000 μg, from about 3 μg to about 1,000 μg, from about 4 μg to about 1,000 μg, from about 5 μg to about 1,000 μg, from about 6 μg to about 1,000 μg, from about 7 μg to about 1,000 μg, from about 8 μg to about 1,000 μg, from about 9 μg to about 1,000 μg, from about 10 μg to about 1,000 μg, from about 11 μg to about 1,000 μg, from about 12 μg to about 1,000 μg, from about 13 μg to about 1,000 μg, from about 14 μg to about 1,000 μg, from about 15 μg to about 1,000 μg, from about 16 μg to about 1,000 μg, from about 17 μg to about 1,000 μg, from about 18 μg to about 1,000 μg, from about 19 μg to about 1,000 μg, from about 20 μg to about 1,000 μg, from about 30 μg to about 1,000 μg, from about 40 μg to about 1,000 μg, from about 50 μg to about 1,000 μg, from about 60 μg to about 1,000 μg, from about 70 μg to about 1,000 μg, from about 80 μg to about 1,000 μg, from about 90 μg to about 1,000 μg, from about 100 μg to about 1,000 μg, from about 200 μg to about 1,000 μg, from about 300 μg to about 1,000 μg, from about 400 μg to about 1,000 μg, from about 500 μg to about 1,000 μg, from about 600 μg to about 1,000 μg, from about 700 μg to about 1,000 μg, from about 800 μg to about 1,000 μg, from about 900 μg to about 1,000 μg, from about 0.1 μg to about 100 μg, from about 1 μg to about 100 μg, from about 1 μg to about 90 μg, from about 1 μg to about 80 μg, from about 1 μg to about 70 μg, from about 1 μg to about 60 μg, from about 1 μg to about 50 μg, from about 1 μg to about 40 μg, from about 1 μg to about 30 μg, from about 1 μg to about 20 μg, from about 1 μg to about 15 μg, from about 1 μg to about 10 μg, or from about 5 μg to about 15 μg NAD(P) can be present in a sensing layer. In some embodiments, NAD(P) can be present in a sensing layer in an amount from about 0.1 μg to about 100 μg.

In some embodiments, the amount of NAD(P) present in the sensing layer can vary depending on the lifetime of the analyte sensor. In some embodiments, the amount of NAD(P) in the sensing layer can allow the analyte sensor to detect an analyte using an NAD(P)-dependent enzyme for at least about 7 days, for at least about 8 days, for at least about 9 days, for at least about 10 days, for at least about 11 days, for at least about 12 days, for at least about 13 days, for at least about 14 days, for at least about 15 days, for at least about 16 days, for at least about 17 days, for at least about 18 days, for at least about 19 days, for at least about 20 days, for at least about 25 days, for at least about 30 days, for at least about 35 days, or for at least about 40 days. In some embodiments, the amount of NAD(P) in the sensing layer can allow the analyte sensor to detect an analyte using an NAD(P)-dependent enzyme for at least about 14 days. In some embodiments, the amount of NAD(P) in the sensing layer can allow the analyte sensor to detect an analyte using an NAD(P)-dependent enzyme for greater than about two weeks, for greater than about three weeks, for greater than about four weeks, for greater than about five weeks, for greater than about six weeks, for greater than about seven weeks, or for greater than about eight weeks.

### 4. Multilayered Membrane Comprising at Least One Negatively Charged Polymer

In some embodiments, the sensing layer is overcoated with a multilayered membrane, wherein the membrane comprises at least one negatively charged polymer. In some embodiments, at least about 10%, at least about 20%, at least about 30%, at least about 40%, at least about 50%, at least about 60%, at least about 70%, at least about 80%, at least about 90% or at least about 95% of the sensing layer can be overcoated with a multilayered membrane comprising at least one negatively charged polymer. In some embodiments, the sensing layer can be entirely overcoated with a multilayered membrane comprising at least one negatively charged polymer. In some embodiments, the multilayered membrane comprising at least one negatively charged polymer limits NAD(P) release. The composition of the multilayered membrane can vary depending on the desired release kinetics of the NAD(P), e.g., rate of NAD(P) release, from the sensing layer.

In some embodiments, the cofactor, e.g., NAD(P), can be physically retained within the sensing layer. For example, but not by way of limitation, a multilayered membrane comprising at least one negatively charged polymer overcoating the sensing layer can aid in retaining the cofactor within the sensing layer while still permitting sufficient inward diffusion of the analyte to permit detection thereof.

In some embodiments, the negatively charged polymer limits transport of at least 70% of the supply of NAD(P) from the sensing layer over a period of 12 days. In some embodiments, the negatively charged polymer limits transport of between 60% and 99% of the supply of NAD(P) from the sensing layer over a period of 12 days. In some embodiments, the negatively charged polymer limits transport of between 60% and 99%, between 60% and 95%, between 60% and 90%, between 60% and 85%, between 60% and 80%, between 60% and 70%, between 70% and 99%, between 70% and 95%, between 70% and 90%, between 70% and 85%, between 70% and 80%, between 80% and 99%, between 80% and 95%, between 80% and 90%, between 80% and 85%, between 85% and 99%, between 85% and 95%, between 85% and 90%, between 90% and 99%, between 90% and 95%, or between 95% and 99% of the supply of NAD(P) from the sensing layer over a period of 12 days. In some embodiments, the negatively charged polymer limits transport of 99%, 95%, 90%, 85%, 80%, 70%, or 60% of the supply of NAD(P) from the sensing layer over a period of 12 days.

The multilayered membrane can be applied over the sensing layer(s) by placing a droplet or droplets of membrane solution on at least the one or more sensing layers of an analyte sensor, such as by dipping the implantable portion into a membrane solution, by spraying the membrane solution on the implantable portion, by heat pressing or melting the membrane solution, vapor depositing the membrane solution, powder coating the membrane solution, or combinations thereof.

Generally, the thickness of the multilayered membrane can be controlled by the number of different membrane solutions, the concentration of the membrane solution(s), by the number of droplets of the membrane solution(s) applied, by the number of times the implantable portion is dipped in the membrane solution(s), by the volume of membrane solution(s) sprayed on the implantable portion, or by any combination thereof. In some embodiments, the multilayered membrane can have a thickness of less than about 100 μm. In some embodiments, the multilayered membrane can have a thickness of less than about 50 μm. In some embodiments, the multilayered membrane can have a thickness ranging from about 0.1 μm to about 100 μm, from about 0.1 μm to about 50 μm, from about 0.1 μm to about 40 μm, from about 0.1 μm to about 20 μm, from about 0.1 μm to about 10 μm, from about 0.1 μm to about 5 μm, from about 0.1 μm to about 1 μm, from about 1 μm to about 100 μm, from about 1 μm to about 50 μm, from about 1 μm to about 40 μm, from about 1 μm to about 20 μm, from about 1 μm to about 10 μm, from about 1 μm to about 5 μm, from about 5 μm to about 100 μm, from about 5 μm to about 50 μm, from about 5 μm to about 40 μm, from about 5 μm to about 20 μm, from about 5 μm to about 10 μm, from about 10 μm to about 100 μm, from about 10 μm to about 50 μm, from about 10 μm to about 40 μm, from about 10 μm to about 20 μm, from about 20 μm to about 100 μm, from about 20 μm to about 50 μm, from about 20 μm to about 40 μm, from about 40 μm to about 100 μm, from about 40 μm to about 50 μm, or from about 50 μm to about 100 μm. In some embodiments, the multilayered membrane can have a thickness of less than about 100 μm, less than about 50 μm, less than about 40 μm, less than about 20 μm, less than about 10 μm, less than about 5 μm, or less than about 1 μm. For example, but not by way of limitation, a sensor (or working electrode) of the present disclosure can be dipped in a membrane solution, or in each different membrane solution if multiple membrane solutions are used, at least once, at least twice, at least three times, at least four times, or at least five times to obtain the desired multilayered membrane thickness.

In some embodiments, the multilayered membrane can be single-component (contains a single membrane polymer). In some embodiments, the multilayered membrane can be multi-component (contains two or more different membrane polymers). In some embodiments, the multilayered membrane can comprise 1, 2, 3, 4, 5, 6, 7, or 8 different membrane polymers. In some embodiments, the multilayered membrane can comprise three different membrane polymers.

In some embodiments, at least one component of the multilayered membrane is a negatively charged polymer. In some embodiments, the multilayered membrane can comprise 1, 2, or different negatively charged polymers. In some embodiments, the multilayered membrane can comprise one negatively charged polymer.

In some embodiments, the multilayered membrane can further comprise at least one polymer that is not negatively charged. In some embodiments, the multilayered membrane can comprise 1, 2, 3, 4, 5, or 6 different polymers that are not negatively charged. In some embodiments, the multilayered membrane can comprise three polymers that are not negatively charged.

In some embodiments, the multilayered membrane can comprise one negatively charged polymer and two different polymers that are not negatively charged.

In some embodiments, the multilayered membrane can include two or more layers. In some embodiments, each layer can be formed by depositing a membrane solution upon a surface, for example by dipping, and allowing the membrane solution to dry. Thus, a first layer can be formed on a sensing layer by dipping an implantable portion into a membrane solution followed by allowing the membrane solution to dry. Then, a second layer can be formed on the first layer by dipping the implantable portion into a membrane solution followed by allowing the membrane solution to dry. Subsequent layers can be formed after each membrane solution is allowed to dry.

In some embodiments, the multilayered membrane can comprise from 1 layer to 8 layers of negatively charged polymer. In some embodiments, the multilayered membrane can comprise from 1 layer to 8 layers, from 1 layer to 6 layers, from 1 layer to 4 layers, from 1 layer to 2 layers, from 2 layers to 8 layers, from 2 layers to 6 layers, from 2 layers to 4 layers, from 4 layers to 8 layers, from 4 layers to 6 layers, or from 6 layers to 8 layers of negatively charged polymer. In some embodiments, the multilayered membrane can comprise 1, 2, 3, 4, 5, 6, 7, or 8 layers of negatively charged polymer. In some embodiments, the multilayered membrane can comprise four layers of negatively charged polymer.

In some embodiments, the multilayered membrane can comprise from 2 layers to 17 layers of polymer that is not negatively charged. In some embodiments, the multilayered membrane can comprise from 1 layers to 17 layers, from 1 layers to 14 layers, from 1 layers to 12 layers, from 1 layers to 10 layers, from 1 layers to 8 layers, from 1 layers to 6 layers, from 1 layers to 4 layers, from 2 layers to 17 layers, from 2 layers to 14 layers, from 2 layers to 12 layers, from 2 layers to 10 layers, from 2 layers to 8 layers, from 2 layers to 6 layers, from 2 layers to 4 layers, from 4 layers to 17 layers, from 4 layers to 14 layers, from 4 layers to 12 layers, from 4 layers to 10 layers, from 4 layers to 8 layers, from 4 layers to 6 layers, from 6 layers to 17 layers, from 6 layers to 14 layers, from 6 layers to 12 layers, from 6 layers to 10 layers, from 6 layers to 8 layers, from 8 layers to 17 layers, from 8 layers to 14 layers, from 8 layers to 12 layers, from 8 layers to 10 layers, from 10 layers to 17 layers, from 10 layers to 16 layers, from 10 layers to 14 layers, from 10 layers to 12 layers, from, from 12 layers to 17 layers, from 12 layers to 14 layers, or from 14 layers to 17 layers of polymer that is not negatively charged. In some embodiments, the multilayered membrane can comprise 1, 2, 3, 4, 5, 6, 7, 8, 9, 10, 11, 12, 13, 14, 15, 16, or 17 layers of polymer that is not negatively charged. In some embodiments, the multilayered membrane can comprise eight layers of polymer that is not negatively charged.

In some embodiments, the multilayered membrane can comprise at least one negatively charged polymer. In some embodiments, the negatively charged polymer can comprise negatively charged sulfonate groups.

In some embodiments the negatively charged polymer can comprise a sulfonic acid or a sulfonic acid salt. In some embodiments, the negatively charged polymer can comprise a perfluorosulfonic acid, a sulfo-phenylated polyphenylene, a styrene sulfonate, or combinations thereof. In some embodiments, the negatively charged polymer can comprise a poly(perflourosulfonic acid), a poly(sulfo-phenylated polyphenylene), a poly(styrene sulfonate), or combinations thereof.

In some embodiments, the negatively charged polymer can comprise a copolymer of poly(tetrafluoroethylene) and a poly(perfluorosulfonic acid).

In some embodiments, the negatively charged polymer can comprise a copolymer of tetrafluoroethylene and a perfluorosulfonic acid having repeat units represented by Formula (I):

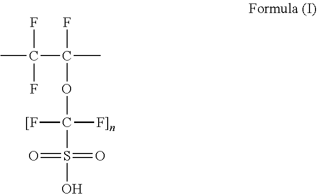

wherein n is an integer from 2 to 4.

In some embodiments, the negatively charged polymer can comprise AQUIVION®.

The use of fluoropolymers is being more strictly regulated and the ability to use these compounds has been removed from certain industries. For this reason, replacements for fluoropolymers have been pursued.

In some embodiments, the negatively charged polymer can comprise a sulfo-phenylated polyphenylene.

In some embodiments, the negatively charged polymer can comprise a sulfo-phenylated polyphenylene polymer having repeat units represented by Formula (II):

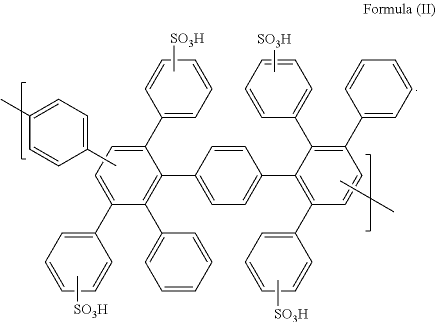

In some embodiments, the negatively charged polymer can comprise PEMION®. In some embodiments, the negatively charged polymer can comprise PEMION® PP1-HNN8-00-X ionomer. In some embodiments, the negatively charged polymer can comprise PEMION® PP1-HNN9-00-X ionomer.

In some embodiments, the negatively charged polymer can comprise a copolymer of poly(vinylpyridine) and poly(styrene sulfonate).

In some embodiments, the negatively charged polymer can comprise poly(4-vinylpyridine-co-4-styrene sulfonic acid salt).

In some embodiments, the negatively charged polymer can comprise poly(4-vinylpyridine)-co-4-styrene sulfonic acid salt represented by Formula (III):

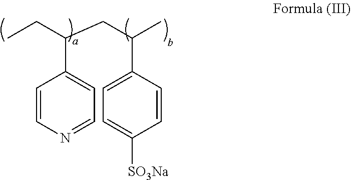

wherein a is an integer from 40 to 100 and b is an integer from 1 to 60.

In some embodiments, in the polymer of Formula (III), a is an integer from 40 to 100, from 40 to 80, from 40 to 70, from 40 to 60, from 40 to 50, from 50 to 100, from 50 to 90, from 50 to 80, from 50 to 70, from 50 to 60, from 60 to 100, from 60 to 90, from 60 to 80, from 60 to 70, from 70 to 100, from 70 to 90, from 70 to 80, from 80 to 100, from 80 to 90, or from 90 to 100.

In some embodiments, in the polymer of Formula (III), b is an integer from 1 to 60, from 1 to 50, from 1 to 40, from 1 to 30, from 1 to 20, from 1 to 10, from 10 to 60, from 10 to 50, from 10 to 40, from 10 to 30, from 10 to 20, from 20 to 60, from 20 to 50, from 20 to 40, from 20 to 30, from 30 to 60, from 30 to 50, from 30 to 40, from 40 to 60, from 40 to 50, or from 50 to 60.

In some embodiments, the multilayered membrane comprising at least one negatively charged polymer can further comprise crosslinked polymers containing heterocyclic nitrogen groups. In some embodiments, a multilayered membrane comprising at least one negatively charged polymer can further comprise a polyvinylpyridine-based polymer. Non-limiting examples of polyvinylpyridine-based polymers are disclosed in U.S. Patent Publication No. 2003/0042137 (e.g., Formula 2b therein), the contents of which are incorporated by reference in their entirety.

In some embodiments, the multilayered membrane comprising at least one negatively charged polymer can further comprise a polyvinylpyridine (e.g., poly(4-vinylpyridine) or poly(4-vinylpyridine)), a polyvinylimidazole, a polyvinylpyridine copolymer (e.g., a copolymer of vinylpyridine and styrene), a polyacrylate, a polyurethane, a polyether urethane, a polyacrylamide, a silicone, a polytetrafluoroethylene, a polyethylene-co-tetrafluoroethylene, a polyolefin, a polyester, a polycarbonate, a biostable polytetrafluoroethylene, homopolymers, copolymers or terpolymers of polyurethanes, a polypropylene, a polyvinylchloride, a polyvinylidene difluoride, a polybutylene terephthalate, a polymethylmethacrylate, a polyether ether ketone, cellulosic polymers, polysulfones and block copolymers thereof including, for example, di-block, tri-block, alternating, random and graft copolymers or a chemically related material and the like.

In some embodiments, the multilayered membrane comprising at least one negatively charged polymer for use in the present disclosure can further comprise a polyvinylpyridine (e.g., poly(4-vinylpyridine) and/or poly(2-vinylpyridine)). In some embodiments, a multilayered membrane comprising at least one negatively charged polymer for use in the present disclosure can further comprise poly(4-vinylpyridine). In some embodiments, a multilayered membrane comprising at least one negatively charged polymer for use in the present disclosure, can further comprise a copolymer of vinylpyridine and styrene. In some embodiments, the multilayered membrane comprising at least one negatively charged polymer can further comprise a polyvinylpyridine-co-styrene copolymer. For example, but not by way of limitation, a polyvinylpyridine-co-styrene copolymer for use in the present disclosure can include a polyvinylpyridine-co-styrene copolymer in which a portion of the pyridine nitrogen atoms are functionalized with a non-crosslinked polyethylene glycol tail and a portion of the pyridine nitrogen atoms are functionalized with an alkylsulfonic acid group. In some embodiments, a derivatized polyvinylpyridine-co-styrene copolymer for use as a membrane polymer can be the osmium-containing poly(4-vinylpyridine)-based polymer as described in U.S. Pat. No. 8,761,857, the contents of which are hereby incorporated by reference in their entirety. In some embodiments, the polyvinylpyridine-based polymer can have a molecular weight from about 50 Da to about 500 kDa.

In some embodiments, the multilayered membrane comprising at least one negatively charged polymer can further comprise polymers such as, but not limited to, poly(styrene co-maleic anhydride), dodecylamine and poly(propylene glycol)-block-polyethylene glycol)-block-poly(propylene glycol) (2-aminopropyl ether) crosslinked with poly(propylene glycol)-block-poly(ethylene glycol)-block-poly(propylene glycol) bis(2-aminopropyl ether); poly(N-isopropyl acrylamide); a copolymer of poly(ethylene oxide) and poly(propylene oxide); or a combination thereof.

In some embodiments, the multilayered membrane comprising at least one negatively charged polymer can further comprise a polyurethane membrane that includes both hydrophilic and hydrophobic regions. In some embodiments, a hydrophobic polymer component can be a polyurethane, a polyurethane urea or poly(ether-urethane-urea). In some embodiments, a polyurethane can be a polymer produced by the condensation reaction of a diisocyanate and a difunctional hydroxyl-containing material. In some embodiments, the polyurethane urea can be a polymer produced by the condensation reaction of a diisocyanate and a difunctional amine-containing material. In some embodiments, diisocyanates for use herein include aliphatic diisocyanates, e.g., diisocyanates comprising from about 4 to about 8 methylene units, or diisocyanates comprising cycloaliphatic moieties. Additional non-limiting examples of polymers that can be used for the generation of a multilayered membrane comprising at least one negatively charged polymer of a presently disclosed sensor include vinyl polymers, polyethers, polyesters, polyamides, inorganic polymers (e.g., polysiloxanes and polycarbosiloxanes), natural polymers (e.g., cellulosic and protein based materials) and mixtures (e.g., admixtures or layered structures) or combinations thereof. In some embodiments, the hydrophilic polymer component can be polyethylene oxide and/or polyethylene glycol. In some embodiments, the hydrophilic polymer component can be a polyurethane copolymer. For example, but not by way of limitation, a hydrophobic-hydrophilic copolymer component for use in the present disclosure can be a polyurethane polymer that comprises about 10% to about 50%, e.g., 20%, hydrophilic polyethylene oxide.

In some embodiments, the multilayered membrane comprising at least one negatively charged polymer can further comprise a silicone polymer/hydrophobic-hydrophilic polymer blend. In some embodiments, the hydrophobic-hydrophilic polymer for use in the blend can be any suitable hydrophobic-hydrophilic polymer such as, but not limited to, polyvinylpyrrolidone, polyhydroxyethyl methacrylate, polyvinylalcohol, polyacrylic acid, polyethers such as polyethylene glycol or polypropylene oxide, and copolymers thereof, including, for example, di-block, tri-block, alternating, random, comb, star, dendritic, and graft copolymers. In some embodiments, the hydrophobic-hydrophilic polymer is a copolymer of poly(ethylene oxide) (PEO) and poly(propylene oxide) (PPO). Non-limiting examples of PEO and PPO copolymers include PEO-PPO diblock copolymers, PPO-PEO-PPO triblock copolymers, PEO-PPO-PEO triblock copolymers, alternating block copolymers of PEO-PPO, random copolymers of ethylene oxide and propylene oxide and blends thereof. In some embodiments, the copolymers can be substituted with hydroxy substituents.

In some embodiments, hydrophilic or hydrophobic modifiers can be used to “fine-tune” the permeability of the resulting multilayered membrane comprising at least one negatively charged polymer to an analyte of interest. In some embodiments, hydrophilic modifiers such as poly(ethylene) glycol, hydroxyl or polyhydroxyl modifiers and the like, and any combinations thereof, can be used to enhance the biocompatibility of the polymer or the resulting multilayered membrane comprising at least one negatively charged polymer.

In some embodiments, the multilayered membrane comprising at least one negatively charged polymer can comprise a membrane polymer crosslinked with a crosslinking agent disclosed herein.

In some embodiments, the multilayered membrane comprising at least one negatively charged polymer can further comprise from 1 layer to 10 layers of poly(4-vinylpyridine). In some embodiments, the multilayered membrane comprising at least one negatively charged polymer can further comprise from 1 layer to 10 layers, from 1 layer to 8 layers, from 1 layer to 6 layers, from 1 layer to 4 layers, from 1 layer to 2 layers, from 2 layers to 10 layers, from 2 layers to 8 layers, from 2 layers to 6 layers, from 2 layers to 4 layers, from 4 layers to 10 layers, from 4 layers to 8 layers, from 4 layers to 6 layers, from 6 layers to 10 layers, from 6 layers to 8 layers, or from 8 layers to 10 layers of poly(4-vinylpyridine). In some embodiments, the multilayered membrane comprising at least one negatively charged polymer can further comprise 1, 2, 3, 4, 5, 6, 7, 8, 9, or 10 layers of poly(4-vinylpyridine). In some embodiments, the multilayered membrane comprising at least one negatively charged polymer can further comprise 6 layers of poly(4-vinylpyridine).

In some embodiments, the multilayered membrane comprising at least one negatively charged polymer can further comprise from 1 layer to 10 layers of osmium-containing poly(4-vinylpyridine)-based polymer. In some embodiments, the multilayered membrane comprising at least one negatively charged polymer can further comprise from 1 layer to 10 layers, from 1 layer to 8 layers, from 1 layer to 6 layers, from 1 layer to 4 layers, from 1 layer to 2 layers, from 2 layers to 10 layers, from 2 layers to 8 layers, from 2 layers to 6 layers, from 2 layers to 4 layers, from 4 layers to 10 layers, from 4 layers to 8 layers, from 4 layers to 6 layers, from 6 layers to 10 layers, from 6 layers to 8 layers, or from 8 layers to 10 layers of osmium-containing poly(4-vinylpyridine)-based polymer. In some embodiments, the multilayered membrane comprising at least one negatively charged polymer can further comprise 1, 2, 3, 4, 5, 6, 7, 8, 9, or 10 layers of osmium-containing poly(4-vinylpyridine)-based polymer. In some embodiments, the multilayered membrane comprising at least one negatively charged polymer can further comprise 2 layers of osmium-containing poly(4-vinylpyridine)-based polymer.

### 5. Enzymes

The sensors of the present disclosure include one or more enzymes for detecting one or more analytes in at least one sensing layer. Suitable enzymes for use in a sensor of the present disclosure include, but are not limited to, any NAD(P)-dependent enzyme. For example, an NAD(P)-dependent enzyme for use in the present disclosure can be used for detecting glucose, ketones, lactate, oxygen, hemoglobin A1C, albumin, alcohol, alkaline phosphatase, alanine transaminase, aspartate aminotransferase, bilirubin, blood, urea, nitrogen, calcium, carbon dioxide, chloride, creatinine, hematocrit, lactate, magnesium, oxygen, pH, phosphorus, potassium, sodium, total protein, uric acid, etc. In some embodiments, the analyte to be detected using an NAD(P)-dependent enzyme can be glucose, lactate, ketones, creatinine, alcohol, e.g., ethanol, or the like. In some embodiments, a sensing layer can include multiple enzymes, e.g., an enzyme system, that are collectively responsive to the analyte.

In some embodiments, the sensing layer of a presently disclosed analyte sensor can include at least one NAD(P)-dependent enzyme. In some embodiments, the sensing layer of a presently disclosed analyte sensor can include two or more NAD(P)-dependent enzymes. In some embodiments, the analyte sensor of the present disclosure can include two sensing layers that each include at least one NAD(P)-dependent enzyme. Alternatively, an analyte sensor of the present disclosure in some embodiments can include two sensing layers, where only one sensing layer includes an NAD(P)-dependent enzyme. Non-limiting examples of NAD(P)-dependent enzymes are disclosed in Vidal et al., Biochimica et Biophysica Acta—Proteins and Proteomics 1866(2):327-347 (2018) (see Tables 1-2), the contents of which are incorporated by reference in their entirety.

In some embodiments, an analyte sensor of the present disclosure can include one or more internal supplies of NAD(P) for an NAD(P)-dependent enzyme included in one or more sensing layers of the analyte sensor.

In some embodiments, a sensing layer can include an NAD(P)-dependent dehydrogenase. Non-limiting examples of NAD(P)-dependent dehydrogenases include glucose dehydrogenase (EC.1.1.1.47), lactate dehydrogenase (EC1.1.1.27 and EC1.1.1.28), malate dehydrogenase (EC1.1.1.37), glycerol dehydrogenase (EC1.1.1.6), alcohol dehydrogenase (EC1.1.1.1), alpha-hydroxybutyrate dehydrogenase, sorbitol dehydrogenase, amino acid dehydrogenase such as L-amino acid dehydrogenase (EC1.4.1.5), diaphorase (EC1.8.1.4), and combinations thereof.

In some embodiments, the NAD(P)-dependent dehydrogenase can include diaphorase, glucose dehydrogenase, alcohol dehydrogenase, lactate dehydrogenase, and β-hydroxybutyrate dehydrogenase. In some embodiments, the enzyme system can include two or more NAD(P)-dependent dehydrogenases, e.g., a first NAD(P)-dependent dehydrogenase and diaphorase. For example, but not by way of limitation, the NAD(P)-dependent dehydrogenase can convert the analyte and oxidized nicotinamide adenine dinucleotide (NAD⁺) into an oxidized analyte and reduced nicotinamide adenine dinucleotide (NADH), respectively. The enzyme cofactors NAD⁺ and NADH aid in promoting the concerted enzymatic reactions disclosed herein. The NADH can then undergo reduction under diaphorase mediation, with the electrons transferred during this process providing the basis for analyte detection at the working electrode.

In some embodiments, an analyte sensor of the present disclosure can include a glucose-responsive sensing layer, a ketones-responsive sensing layer, a lactate-responsive sensing layer, a creatinine-responsive sensing layer, an alcohol-responsive sensing layer, or any combination thereof. In some embodiments, a glucose-responsive sensing layer can include one or more NAD(P)-dependent enzymes for detecting glucose. In some embodiments, a ketones-responsive sensing layer can include one or more NAD(P)-dependent enzymes for detecting ketones. In some embodiments, a lactate-responsive sensing layer can include one or more NAD(P)-dependent enzymes for detecting lactate. In some embodiments, a creatinine-responsive sensing layer can include one or more NAD(P)-dependent enzymes for detecting creatinine. In some embodiments, an alcohol-responsive sensing layer can include one or more NAD(P)-dependent enzymes for detecting alcohol. In some embodiments, a sensing layer can include an enzyme system comprising two or more enzymes that are collectively responsive to the analyte. For example, but not by way of limitation, a ketones-responsive sensing layer can include an enzyme system comprising at least one NAD(P)-dependent enzyme.

In some embodiments, a sensing layer can be a glucose-responsive sensing layer that includes at least one NAD(P)-dependent enzyme for detecting glucose. In some embodiments, the glucose-responsive sensing layer can include a glucose dehydrogenase. For example, but not by way of limitation, an analyte sensor of the present disclosure for detecting glucose can have a sensing layer comprising a supply of NAD(P) and an enzyme system that includes glucose dehydrogenase.

In some embodiments, a sensing layer can be an alcohol-responsive sensing layer that includes at least one NAD(P)-dependent enzyme for detecting one or more alcohols. In some embodiments, the alcohol-responsive sensing layer can include an alcohol dehydrogenase. For example, but not by way of limitation, an analyte sensor of the present disclosure for detecting an alcohol has a sensing layer comprising a supply of NAD(P) and an enzyme system that includes alcohol dehydrogenase.

For example, but not by way of limitation, an alcohol-responsive sensing layer can include an alcohol dehydrogenase as shown in FIG. 6. In certain embodiments, a sensing layer containing an alcohol dehydrogenase (“ADH” in FIG. 6) can convert alcohol and NAD(P) into an aldehyde (e.g., acetaldehyde) and reduced nicotinamide adenine dinucleotide phosphate (NAD(P)H), respectively. In FIG. 6, diaphorase is chemically bound (e.g., covalently) to a polymer that is disposed upon a working electrode (i.e., in a sensing layer of an alcohol sensor) and ADH is chemically bound (e.g., covalently) to the same (e.g., via a crosslinker) or separate polymer. In one embodiment, diaphorase can be chemically bound to a first polymer that is disposed upon a working electrode and ADH can be chemically bound to a second polymer that is disposed upon a second polymer that is disposed upon the first polymer. In addition to diaphorase and/or ADH, an osmium complex or other transition metal complex capable of exchanging electrons with the enzyme (e.g., a derivatized polyvinylpyridine-co-styrene copolymer (“D-PVP” in FIG. 6)) is also covalently (e.g., covalently) bound to the polymer that is disposed upon the working electrode. A derivatized polyvinylpyridine-co-styrene copolymer comprises both covalently bound polymer and electron transfer agent. In some embodiments, the sensing layer comprises a derivatized polyvinylpyridine-co-styrene polymer, to which diaphorase and ADH are chemically bound, and are overcoated with a membrane.

In some embodiments, a sensing layer can be a ketones-responsive sensing layer that includes at least one NAD(P)-dependent enzyme for detecting one or more ketones. In some embodiments, the ketones-responsive sensing layer can include p-hydroxybutyrate dehydrogenase. For example, but not by way of limitation, an analyte sensor of the present disclosure for detecting ketones can have a sensing layer comprising a supply of NAD(P) and an enzyme system that includes β-hydroxybutyrate dehydrogenase.

In some embodiments, a sensing layer can be a lactate-responsive sensing layer that includes at least one NAD(P)-dependent enzyme for detecting lactate. For example, but not by way of limitation, a lactate-responsive sensing layer can include a lactate dehydrogenase. In some embodiments an analyte sensor of the present disclosure for detecting lactate has a sensing layer comprising a supply of NAD(P) and an enzyme system that includes a lactate dehydrogenase.

In some embodiments, an analyte sensor disclosed herein can include at least one sensing layer that includes one or more NAD(P)-dependent enzymes, as disclosed herein, for detecting an analyte. Alternatively, an analyte sensor disclosed herein can include two or more sensing layers, with each sensing layer containing one or more enzymes, e.g., where at least one of the sensing layers includes one or more NAD(P)-dependent enzymes. For example, but not by way of limitation, an analyte sensor of the present disclosure can include a first sensing layer that comprises a first enzyme (or enzyme system) for use in detecting a first analyte and a second sensing layer that includes a second enzyme (or second enzyme system) for detecting a second analyte, where at least the first sensing layer or second sensing layer includes an NAD(P)-dependent enzyme.

In some embodiments, the sensing layer can include by weight from about 10% to about 80%, e.g., from about 15% to about 75%, from about 20% to about 70%, from about 25% to about 65%, or from about 30% to about 60%, of one or more enzymes (e.g., one or more NAD(P)-dependent enzymes) disclosed herein.

In some embodiments, the sensing layer can further include a stabilizer, e.g., for stabilizing the enzyme. For example, but not by way of limitation, the stabilizer can be an albumin, e.g., a serum albumin. Non-limiting examples of serum albumins include bovine serum albumin and human serum albumin. In some embodiments, the stabilizer is a human serum albumin. In some embodiments, the stabilizer can be a bovine serum albumin. In some embodiments, the stabilizer can be catalase. In some embodiments, the sensing layer can include a ratio of stabilizer to the one or more enzymes present in the sensing layer, e.g., NAD(P)-dependent enzyme, from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In some embodiments, the sensing layer can include a ratio of stabilizer to the one or more enzymes present in the sensing layer, e.g., NAD(P)-dependent enzyme, from about 2:1 to about 1:2. In some embodiments, the sensing layer can include a ratio of stabilizer to the NAD(P)-dependent enzyme, e.g., NAD(P)-dependent dehydrogenase, from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In some embodiments, the sensing layer can include a ratio of stabilizer to the NAD(P)-dependent enzyme, e.g., NAD(P)-dependent dehydrogenase, from about 2:1 to about 1:2. In some embodiments, the sensing layer can include by weight from about 10% to about 50%, e.g., from about 15% to about 45%, from about 20% to about 40%, from about 20% to about 35%, or from about 20% to about 30% of the stabilizer. In some embodiments, the sensing layer can include from about 15% to about 35% of the stabilizer by weight.

In some embodiments, in addition to the presence of an NAD(P), the sensing layer can further include a cofactor for one or more enzymes present in the sensing layer. In some embodiments, the cofactor is NAD(P). In some embodiments, the cofactor is a cofactor different from NAD(P). In some embodiments, the sensing layer can include a ratio of cofactor to enzyme from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2, or from about 2:1 to about 1:2. In some embodiments, the sensing layer can include a ratio of cofactor to enzyme from about 2:1 to about 1:2. In some embodiments, the sensing layer can include by weight from about 10% to about 50%, e.g., from about 15% to about 45%, from about 20% to about 40%, from about 20% to about 35%, or from about 20% to about 30% of the cofactor. In some embodiments, the sensing layer can include from about 15% to about 35% by weight of the cofactor.

In some embodiments, an analyte sensor of the present disclosure can include an implantable portion comprising at least one working electrode, and a sensing layer disposed upon the surface of the working electrode, where the sensing layer includes at least one NAD(P)-dependent enzyme. In some embodiments, an analyte sensor of the present disclosure can include an implantable portion comprising a substrate, at least one working electrode, and a sensing layer disposed upon the surface of the working electrode, where the sensing layer includes at least one NAD(P)-dependent enzyme. In some embodiments, the NAD(P)-dependent enzyme is an NAD(P)-dependent dehydrogenase. For example, but not by way of limitation, a sensor of the present disclosure can include an implantable portion comprising at least one working electrode and a sensing layer disposed upon the surface of the working electrode, where the sensing layer includes an enzyme system comprising an NAD(P) dependent-dehydrogenase.

In some embodiments, a sensor of the present disclosure can include an implantable portion comprising at least one working electrode and an alcohol-responsive sensing layer disposed upon the surface of the working electrode, where the alcohol-responsive sensing layer includes an enzyme system comprising an NAD(P)-dependent dehydrogenase, e.g., β-hydroxybutyrate dehydrogenase. In some embodiments, the enzyme system further includes diaphorase.

In some embodiments, an analyte sensor of the present disclosure can include a second sensing layer, e.g., for detecting an analyte different from the analyte detected by the first sensing layer. In some embodiments, the second sensing layer is disposed upon the same working electrode as the first sensing layer or on a second working electrode. In some embodiments, the second sensing layer is a glucose-responsive sensing layer, a lactate-responsive sensing layer, a creatinine-responsive sensing layer, or an alcohol-responsive sensing layer.

In some embodiments, the second sensing layer of an analyte sensor of the present disclosure can include one or more enzymes for detecting glucose. For example, but not by way of limitation, an analyte sensor of the present disclosure can include a sensing layer (e.g., a second sensing layer) that comprises one or more enzymes for detecting glucose, e.g., disposed on a second working electrode. In some embodiments, the analyte sensor can include a sensing layer comprising a glucose oxidase and/or a glucose dehydrogenase for detecting glucose.

In some embodiments, the second sensing layer can include one or more enzymes for detecting lactate. For example, but not by way of limitation, an analyte sensor of the present disclosure can include a sensing layer (e.g., a second sensing layer) that comprises one or more enzymes, e.g., an enzyme system, for detecting lactate, e.g., disposed on a second working electrode. In some embodiments, the analyte sensor can include sensing layer comprising a lactate dehydrogenase and/or a lactate oxidase.

In some embodiments, the second sensing layer, e.g., present on a second working electrode, of an analyte sensor of the present disclosure can include one or more enzymes for detecting alcohol. For example, but not by way of limitation, an analyte sensor of the present disclosure can include a sensing layer (e.g., a second sensing layer) that comprises one or more enzymes, e.g., an enzyme system, for detecting alcohol, e.g., disposed on a second working electrode. In some embodiments, the analyte sensor can include a sensing layer comprising an alcohol dehydrogenase.

In some embodiments, the second sensing layer, e.g., present on a second working electrode, of an analyte sensor of the present disclosure can include one or more enzymes for detecting creatinine. For example, but not by way of limitation, an analyte sensor of the present disclosure can include a sensing layer (e.g., a second sensing layer) that comprises one or more enzymes, e.g., an enzyme system, for detecting creatinine, e.g., disposed on a second working electrode. In some embodiments, the analyte sensor can include a sensing area comprising an amidohydrolase, creatine amidinohydrolase, and/or sarcosine oxidase.

In some embodiments, an analyte sensor can include two working electrodes, e.g., a first sensing layer disposed on a first working electrode and a second sensing layer disposed on a second working electrode. For example, but not by way of limitation, an analyte sensor disclosed herein can feature a first sensing layer disposed on a first working electrode and a second sensing layer disposed upon the surface of a different working electrode, e.g., second working electrode, where at least one of the sensing layers includes a supply of NAD(P) and an NAD(P)-dependent enzyme. In some embodiments, the second sensing layer can be configured to detect a different analyte or the same analyte detected by first sensing layer. In some embodiments, such analyte sensors can include an implantable portion with a first working electrode and a second working electrode, a first sensing layer disposed upon a surface of the first working electrode and a second sensing layer disposed upon a surface of the second working electrode, where at least one of the sensing layers includes a supply of NAD(P) and an NAD(P)-dependent enzyme.

In some embodiments, when the sensor is configured to detect two or more analytes using two working electrodes, detection of each analyte can include applying a potential to each working electrode separately, such that separate signals are obtained from each analyte. The signal obtained from each analyte can then be correlated to an analyte concentration through use of a calibration curve or function, or by employing a lookup table. In some embodiments, correlation of the analyte signal to an analyte concentration can be conducted through use of a processor.

In some analyte sensor configurations, the first sensing layer and the second sensing layer can be disposed upon a single working electrode. For example, but not by way of limitation, an analyte sensor disclosed herein can feature a first sensing layer and a second sensing layer disposed upon the surface of a single working electrode, where at least one of the sensing layers includes an supply of NAD(P) and an NAD(P)-dependent enzyme. In some embodiments, a first signal can be obtained from the first sensing layer, e.g., at a low potential, and a second signal containing a signal contribution from both sensing layers can be obtained at a higher potential. Subtraction of the first signal from the second signal can then allow the signal contribution arising from the second analyte to be determined. The signal contribution from each analyte can then be correlated to an analyte concentration in a similar manner to that described for sensor configurations having multiple working electrodes. In some embodiments, when an alcohol-responsive sensing layer and a second sensing layer configured to detect a different analyte, e.g., a glucose-responsive sensing layer, are arranged upon a single working electrode in this manner, one of the sensing layers can be configured such that it can be interrogated separately to facilitate detection of each analyte. For example, either the alcohol-responsive sensing layer or glucose-responsive sensing layer can produce a signal independently of the other sensing layer.

It is also to be appreciated that the sensitivity (output current) of the analyte sensors toward each analyte can be varied by changing the coverage (area or size) of the sensing layers, the area ratio of the sensing layers with respect to one another, or the identity, thickness and/or composition of a mass transport limiting membrane overcoating the sensing layers. Variation of these parameters can be conducted readily by one having ordinary skill in the art once granted the benefit of the disclosure herein.

### 6. Redox Mediators

In some embodiments, an analyte sensor disclosed herein can include an electron transfer agent. For example, but not by way of limitation, one or more sensing layers of an analyte sensor can include an electron transfer agent. In some embodiments, an analyte sensor can include one sensing layer that includes an electron transfer agent and a second sensing layer that does not include an electron transfer agent. In some embodiments, the presence of an electron transfer agent in a sensing layer can depend on the enzyme or enzyme system used to detect the analyte and/or the composition of the working electrode. Alternatively, an analyte sensor can include two sensing layers, where both sensing layers include an electron transfer agent.

Suitable electron transfer agents can facilitate conveyance of electrons to the adjacent working electrode after an analyte undergoes an enzymatic oxidation-reduction reaction within the corresponding sensing layer, thereby generating a current that is indicative of the presence of that particular analyte. The amount of current generated is proportional to the quantity of analyte that is present. In some embodiments, suitable electron transfer agents can include electroreducible and electrooxidizable ions, complexes or molecules (e.g., quinones) having oxidation-reduction potentials that are a few hundred millivolts above or below the oxidation-reduction potential of the standard calomel electrode (SCE). In some embodiments, the redox mediators can include osmium complexes and other transition metal complexes, such as those described in U.S. Pat. Nos. 6,134,461 and 6,605,200, which are incorporated herein by reference in their entirety. Additional examples of suitable redox mediators include those described in U.S. Pat. Nos. 6,736,957, 7,501,053 and 7,754,093, the disclosures of each of which are also incorporated herein by reference in their entirety. Other examples of suitable redox mediators include metal compounds or complexes of ruthenium, osmium, iron (e.g., polyvinylferrocene or hexacyanoferrate), or cobalt, including metallocene compounds thereof, for example. Suitable ligands for the metal complexes can also include, for example, bidentate or higher denticity ligands such as, for example, bipyridine, biimidazole, phenanthroline, or pyridyl(imidazole). Other suitable bidentate ligands can include, for example, amino acids, oxalic acid, acetylacetone, diaminoalkanes, or o-diaminoarenes. Any combination of monodentate, bidentate, tridentate, tetradentate, or higher denticity ligands can be present in a metal complex, e.g., osmium complex, to achieve a full coordination sphere.

In some embodiments, electron transfer agents disclosed herein can comprise suitable functionality to promote covalent bonding to a polymer (also referred to herein as a polymeric backbone) within the sensing layers as discussed further below. For example, but not by way of limitation, an electron transfer agent for use in the present disclosure can include a polymer-bound electron transfer agent. Suitable non-limiting examples of polymer-bound electron transfer agents include those described in U.S. Pat. Nos. 8,444,834, 8,268,143 and 6,605,201, the disclosures of which are incorporated herein by reference in their entirety. In some embodiments, the electron transfer agent is a bidentate osmium complex bound to a polymer described herein, e.g., a polymeric backbone described below. In some embodiments, the polymer-bound electron transfer agent shown in FIG. 3 of U.S. Pat. No. 8,444,834 can be used in a sensor of the present disclosure.

In some embodiments of the present disclosure, an analyte sensor can include at least one working electrode and at least one sensing layer disposed upon the surface of the working electrode, where the sensing layer includes a supply of NAD(P), at least one NAD(P)-dependent enzyme, and at least one redox mediator, e.g., an osmium complex. In some embodiments, the sensing layer includes an enzyme system comprising a diaphorase, an NAD(P)-dependent dehydrogenase, e.g., p-hydroxybutyrate dehydrogenase, and a redox mediator, e.g., an osmium complex.

### 7. Polymeric Backbone

In some embodiments, one or more sensing layers for promoting analyte detection can include a polymer to which an enzyme and/or redox mediator is covalently bound. Any suitable polymeric backbone can be present in the sensing layer for facilitating detection of an analyte through covalent bonding of the enzyme and/or redox mediator thereto. Non-limiting examples of suitable polymers within the sensing layer include polyvinylpyridines, e.g., poly(4-vinylpyridine) and/or poly(2-vinylpyridine), and polyvinylimidazoles, e.g., poly(N-vinylimidazole) and poly(1-vinylimidazole), or a copolymer thereof, for example, in which quaternized pyridine groups serve as a point of attachment for the redox mediator or enzyme thereto. Illustrative copolymers that can be suitable for inclusion in the sensing layers include those containing monomer units such as styrene, acrylamide, methacrylamide, or acrylonitrile, for example. In some embodiments, polymers that can be present in a sensing layer include a polyurethane or a copolymer thereof, and/or polyvinylpyrrolidone. In some embodiments, polymers that can be present in the sensing layer include, but are not limited to, those described in U.S. Pat. No. 6,605,200, the contents of which are incorporated herein by reference in their entirety, such as poly(acrylic acid), styrene/maleic anhydride copolymer, methylvinylether/maleic anhydride copolymer (GANTREZ polymer), poly(vinylbenzylchloride), poly(allylamine), polylysine, poly(4-vinylpyridine) quaternized with carboxypentyl groups, and poly(sodium 4-styrene sulfonate). In some embodiments where the analyte sensor includes two sensing layers, the polymer within each sensing layer can be the same or different.

In some embodiments, the polymer can be polyvinylpyridine or a copolymer thereof. In some embodiments, the polymer can be a co-polymer of vinylpyridine and styrene.

In some embodiments, when an enzyme system with multiple enzymes is present in a given sensing layer, all of the multiple enzymes can be covalently bonded to the polymer. In some embodiments, only a subset of the multiple enzymes are covalently bonded to the polymer. For example, and not by the way of limitation, one or more enzymes within an enzyme system can be covalently bonded to the polymer and at least one enzyme can be non-covalently associated with the polymer, such that the non-covalently bonded enzyme is physically retained within the polymer. In some embodiments, the NAD(P)-dependent enzyme can be covalently bonded to the polymer. Alternatively, the NAD(P)-dependent enzyme can be non-covalently associated with the polymer. In some embodiments, the NAD(P)-dependent dehydrogenase and the diaphorase can be covalently bonded to a polymer within a sensing layer of the disclosed analyte sensors. In some embodiments, the NAD(P)-dependent dehydrogenase can be covalently bonded to the polymer and diaphorase can be non-covalently associated with the polymer. Alternatively, diaphorase can be covalently bonded to the polymer and the NAD(P)-dependent dehydrogenase can be non-covalently associated with the polymer.

In some embodiments, when a stabilizer is present in a sensing layer, one or more enzymes within the area can be covalently bonded to the stabilizer. For example, and not by the way of limitation, one or more enzymes within an enzyme system, e.g., one or more NAD(P)-dependent enzymes, can be covalently bonded to the stabilizer, e.g., albumin, present in the sensing layer.

In some particular embodiments, covalent bonding of the one or more enzymes and/or redox mediators to the polymer and/or stabilizer in a given sensing layer can take place via crosslinking introduced by a suitable crosslinking agent. In some embodiments, crosslinking of the polymer to the one or more enzymes and/or redox mediators can reduce the occurrence of delamination of the enzyme compositions from the electrode. Suitable crosslinking agents can include one or more crosslinkable functionalities such as, but not limited to, vinyl, alkoxy, acetoxy, enoxy, oxime, amino, hydroxyl, cyano, halo, acrylate, epoxide, and isocyanato groups. In some embodiments, the crosslinking agent can comprise one or more, two or more, three or more, or four or more epoxide groups. For example, but not by way of limitation, a crosslinker for use in the present disclosure can include mono-, di-, tri- and tetra-ethylene oxides. In some embodiments, crosslinking agents for reaction with free amino groups in the enzyme (e.g., with the free side chain amine in lysine) can include crosslinking agents such as, for example, polyethylene glycol dibutyl ethers, polypropylene glycol dimethyl ethers, polyalkylene glycol allyl methyl ethers, polyethylene glycol diglycidyl ether (PEGDGE), or other polyepoxides, cyanuric chloride, N-hydroxysuccinimide, imidoesters, epichlorohydrin, or derivatized variants thereof. In some embodiments, the crosslinking agent can be PEGDGE, e.g., having an average molecular weight (Mn) from about 200 to 1,000, e.g., about 400. In some embodiments, the crosslinking agent can be PEGDGE 400. In some embodiments, the crosslinking agent can be glutaraldehyde. Suitable crosslinking agents for reaction with free carboxylic acid groups in the enzyme can include, for example, carbodiimides. In some embodiments, the crosslinking agent can be polyethylene glycol diglycidyl ether. In some embodiments, the crosslinking of the enzyme to the polymer can generally be intermolecular. In some embodiments, the crosslinking of the enzyme to the polymer can generally be intramolecular.

### 8. Mass Transport Limiting Membrane

As used herein, a mass transport limiting membrane is a membrane that does not comprise a negatively charged polymer.

In some embodiments, the analyte sensors disclosed herein further include a mass transport limiting membrane permeable to an analyte that overcoats at least one sensing layer, e.g., a first sensing layer and/or a second sensing layer. In some embodiments, the mass transport limiting membrane overcoats one or more of the sensing layers of an analyte sensor. In some embodiments, a multilayered membrane comprising at least one negatively charged polymer can overcoat the first sensing layer and a mass transport limiting membrane can overcoat the second sensing layer. In some embodiments, a multilayered membrane comprising at least one negatively charged polymer can overcoat more than one sensing layer. In some embodiments, a multilayered membrane comprising at least one negatively charged polymer can overcoat one of the sensing layers and a mass transport limiting membrane can overcoat both the first and second sensing layers.

In some embodiments, a mass transport limiting membrane overcoating a sensing layer can improve biocompatibility. A mass transport limiting membrane can act as a diffusion-limiting barrier to reduce the rate of mass transport of the analyte, e.g., glucose, an alcohol, a ketone, lactate or β-hydroxybutyrate, when the sensor is in use. For example, but not by way of limitation, limiting access of an analyte, e.g., an alcohol, to the sensing layer with a mass transport limiting membrane can aid in avoiding sensor overload (saturation), thereby improving detection performance and accuracy. In some embodiments, the mass transport limiting membrane can limit the flux of an analyte to the electrode in an electrochemical sensor so that the sensor is linearly responsive over a large range of analyte concentrations.

In some embodiments, the mass transport limiting membrane can have a thickness, e.g., dry thickness, ranging from about 0.1 μm to about 1,000 μm, e.g., from about 1 μm to about 500 μm, from about 1 μm to about 100 μm, or from about 10 μm to about 100 μm. In some embodiments, the mass transport limiting membrane can have a thickness from about 0.1 μm to about 10 μm, e.g., from about 0.5 μm to about 10 μm, from about 1 μm to about 10 μm, from about 1 μm to about 5 μm, or from about 0.1 μm to about 5 μm.

In some embodiments, the mass transport limiting membrane can include two or more layers. In some embodiments, each layer can be formed by depositing a mass transport limiting membrane solution upon a surface, for example by dipping, and allowing the membrane solution to dry. In some embodiments, the sensor can be dipped in the mass transport limiting membrane solution more than once. For example, but not by way of limitation, a sensor (or working electrode) of the present disclosure can be dipped in an mass transport limiting membrane solution at least twice, at least three times, at least four times, or at least five times to obtain the desired mass transport limiting membrane thickness.

In some embodiments, the mass transport limiting membrane can be single-component (contain a single membrane polymer). Alternatively, the mass transport limiting membrane can be multi-component (contain two or more different membrane polymers). In some embodiments, the mass transport limiting membrane can include two or more layers, e.g., a bilayer or trilayer membrane. In some embodiments, each layer can comprise a different polymer or the same polymer at different concentrations or thicknesses. In some embodiments, the first sensing layer can be covered by a multilayered membrane comprising at least one negatively charged polymer, and the second sensing layer can be covered by a single mass transport limiting membrane. In some embodiments, the first sensing layer can be covered by a multilayered membrane comprising at least one negatively charged polymer, and the second sensing layer can be covered by a multilayered mass transport limiting membrane. In some embodiments, the first sensing layer can be covered by a multilayered membrane comprising at least one negatively charged polymer and the second sensing layer can be covered by a multilayered membrane comprising at least one negatively charged polymer. In some embodiments, the first sensing layer can be covered by a multilayered membrane comprising at least one negatively charged polymer and the first sensing layer and the second sensing layer can be covered by a single mass transport limiting membrane.

In some embodiments, a mass transport limiting membrane can include a polyvinylpyridine (e.g., poly(4-vinylpyridine) or poly(4-vinylpyridine)), a polyvinylimidazole, a polyvinylpyridine copolymer (e.g., a copolymer of vinylpyridine and styrene), a polyacrylate, a polyurethane, a polyether urethane, a silicone, a polytetrafluoroethylene, a polyethylene-co-tetrafluoroethylene, a polyolefin, a polyester, a polycarbonate, a biostable polytetrafluoroethylene, homopolymers, copolymers or terpolymers of polyurethanes, a polypropylene, a polyvinylchloride, a polyvinylidene difluoride, a polybutylene terephthalate, a polymethylmethacrylate, a polyether ether ketone, cellulosic polymers, polysulfones and block copolymers thereof including, for example, di-block, tri-block, alternating, random and graft copolymers or a chemically related material and the like.

In some embodiments, the mass transport limiting membrane for use in the present disclosure, e.g., a single-component membrane, can include a polyvinylpyridine (e.g., poly(4-vinylpyridine) and/or poly(2-vinylpyridine)). In some embodiments, a mass transport limiting membrane for use in the present disclosure, e.g., a single-component membrane, can include poly(4-vinylpyridine). In some embodiments, a mass transport limiting membrane for use in the present disclosure, e.g., a single-component membrane, can include a copolymer of vinylpyridine and styrene. In some embodiments, the mass transport limiting membrane can comprise a polyvinylpyridine-co-styrene copolymer. For example, but not by way of limitation, a polyvinylpyridine-co-styrene copolymer for use in the present disclosure can include a polyvinylpyridine-co-styrene copolymer in which a portion of the pyridine nitrogen atoms were functionalized with a non-crosslinked polyethylene glycol tail and a portion of the pyridine nitrogen atoms were functionalized with an alkylsulfonic acid group. In some embodiments, a derivatized polyvinylpyridine-co-styrene copolymer for use as a membrane polymer can be the osmium-containing poly(4-vinylpyridine)-based polymer as described in U.S. Pat. No. 8,761,857, the contents of which are incorporated by reference in their entirety. In some embodiments, the osmium-containing poly(4-vinylpyridine-based) polymer can have a molecular weight from about 50 Da to about 500 kDa.

In some embodiments, the mass transport limiting membrane can comprise polymers such as, but not limited to, poly(styrene co-maleic anhydride), dodecylamine and poly(propylene glycol)-block-polyethylene glycol)-block-poly(propylene glycol) (2-aminopropyl ether) crosslinked with poly(propylene glycol)-block-poly(ethylene glycol)-block-poly(propylene glycol) bis(2-aminopropyl ether); poly(N-isopropyl acrylamide); a copolymer of poly(ethylene oxide) and poly(propylene oxide); or a combination thereof.

In some embodiments, the mass transport limiting membrane can include a polyurethane membrane that includes both hydrophilic and hydrophobic regions. In some embodiments, a hydrophobic polymer component can be a polyurethane, a polyurethane urea or poly(ether-urethane-urea). In some embodiments, a polyurethane is a polymer produced by the condensation reaction of a diisocyanate and a difunctional hydroxyl-containing material. In some embodiments, a polyurethane urea is a polymer produced by the condensation reaction of a diisocyanate and a difunctional amine-containing material. In some embodiments, diisocyanates for use herein can include aliphatic diisocyanates, e.g., containing from about 4 to about 8 methylene units, or diisocyanates containing cycloaliphatic moieties. Additional non-limiting examples of polymers that can be used for the generation of a mass transport limiting membrane of the presently disclosed sensor include vinyl polymers, polyethers, polyesters, polyamides, inorganic polymers (e.g., polysiloxanes and polycarbosiloxanes), natural polymers (e.g., cellulosic and protein based materials) and mixtures (e.g., admixtures or layered structures) or combinations thereof. In some embodiments, the hydrophilic polymer component can be polyethylene oxide and/or polyethylene glycol. In some embodiments, the hydrophilic polymer component can be a polyurethane copolymer. For example, but not by way of limitation, a hydrophobic-hydrophilic copolymer component for use in the present disclosure can be a polyurethane polymer that comprises about 10% to about 50%, e.g., 20%, hydrophilic polyethylene oxide.

In some embodiments, the mass transport limiting membrane can include a silicone polymer/hydrophobic-hydrophilic polymer blend. In some embodiments, the hydrophobic-hydrophilic polymer for use in the blend can be any suitable hydrophobic-hydrophilic polymer such as, but not limited to, polyvinylpyrrolidone, polyhydroxyethyl methacrylate, polyvinylalcohol, polyacrylic acid, polyethers such as polyethylene glycol or polypropylene oxide, and copolymers thereof, including, for example, di-block, tri-block, alternating, random, comb, star, dendritic, and graft copolymers. In some embodiments, the hydrophobic-hydrophilic polymer can be a copolymer of poly(ethylene oxide) (PEO) and poly(propylene oxide) (PPO). Non-limiting examples of PEO and PPO copolymers include PEO-PPO diblock copolymers, PPO-PEO-PPO triblock copolymers, PEO-PPO-PEO triblock copolymers, alternating block copolymers of PEO-PPO, random copolymers of ethylene oxide and propylene oxide and blends thereof. In some embodiments, the copolymers can be substituted with hydroxy substituents.

In some embodiments, hydrophilic or hydrophobic modifiers can be used to “fine-tune” the permeability of the resulting membrane to an analyte of interest. In some embodiments, hydrophilic modifiers such as poly(ethylene) glycol, hydroxyl or polyhydroxyl modifiers and the like, and any combinations thereof, can be used to enhance the biocompatibility of the polymer or the resulting mass transport limiting membrane.

In some embodiments where multiple sensing layers are present, the mass transport limiting membrane can overcoat each sensing layer, including the option of overcoating a sensing layer coated with a multilayered membrane comprising at least one negatively charged polymer, which can be achieved by dip coating operations to produce a mass transport limiting membrane portion upon a the multilayered membrane comprising at least one negatively charged polymer.

In some embodiments where multiple sensing layers are present, a separate mass transport limiting membrane can overcoat each sensing layer, including a sensing layer already overcoated with a multilayered membrane comprising at least one negatively charged polymer. For example, but not by way of limitation, a mass transport limiting membrane can be disposed on the first sensing layer, e.g., an alcohol-responsive sensing layer, and a separate, second mass transport limiting membrane can overcoat the second sensing layer, e.g., a glucose-responsive sensing layer. In some embodiments, the two mass transport limiting membranes can be spatially separated and do not overlap each other. In some embodiments, the first mass transport limiting membrane does not overlap the second mass transport limiting membrane and the second mass transport limiting membrane does not overlap the first mass transport limiting membrane. In some embodiments, the first mass transport limiting membrane comprises different polymers than the second mass transport limiting membrane. Alternatively, the first mass transport limiting membrane comprises the same polymers as the second mass transport limiting membrane. In some embodiments, the first mass transport limiting membrane comprises the same polymers as the second mass transport limiting membrane but comprises different crosslinking agents.

In some embodiments, polydimethylsiloxane (PDMS) can be incorporated in any of the mass transport limiting membranes disclosed herein.

In some embodiments, an analyte sensor described herein can comprise an implantable portion comprising a first working electrode, a first sensing layer disposed upon a surface of the first working electrode, wherein the first sensing layer comprises an NAD(P)-dependent enzyme and a supply of NAD(P), a multilayered membrane that overcoats the sensing layer comprising at least one negatively charged polymer, and a mass transport limiting membrane permeable to the first analyte that overcoats at least the first sensing layer (and also overcoats the multilayered membrane comprising at least one negatively charged polymer). In some embodiments, the first sensing layer is not further overcoated with a mass transport limiting membrane.

In some embodiments, the first sensing layer can comprise a first polymer and an enzyme responsive, e.g., an NAD(P)-dependent enzyme, to a first analyte, e.g., alcohol, that is, optionally, covalently bonded to a first polymer. For example, but not by way of limitation, an analyte sensor described herein can comprise an implantable portion comprising a first working electrode, a first alcohol-responsive sensing layer disposed upon a surface of the first working electrode, wherein the first sensing layer comprises an NAD(P)-dependent enzyme and a supply of NAD(P), a multilayered membrane that overcoats the alcohol-responsive area comprising at least one negatively charged polymer, and a mass transport limiting membrane permeable to the first analyte that overcoats at least the first sensing layer. In some embodiments, the first alcohol-responsive sensing layer is not further overcoated with a mass transport limiting membrane.

In some embodiments, the first sensing layer can comprise a first polymer and an enzyme system responsive to a first analyte, e.g., an alcohol, that comprises at least one enzyme, e.g., an NAD-dependent enzyme, that is, optionally, covalently bonded to the first polymer. For example, but not by way of limitation, an analyte sensor described herein can comprise an implantable portion comprising a first working electrode, a first alcohol-responsive sensing layer comprising an enzyme system comprising alcohol dehydrogenase and diaphorase (where one or both enzymes are covalently bonded to a polymer) disposed upon a surface of the first working electrode, wherein the first sensing layer comprises an NAD(P)-dependent enzyme and a supply of NAD(P), a multilayered membrane that overcoats the alcohol-responsive area comprising at least one negatively charged polymer, and further comprising a mass transport limiting membrane permeable to the first analyte that overcoats at least the first sensing layer. In some embodiments, the first alcohol-responsive sensing layer is not further overcoated with a mass transport limiting membrane.

In some embodiments when a first sensing layer and a second sensing layer configured for assaying different analytes are disposed on separate working electrodes, the mass transport limiting membrane can have differing permeability values for the first analyte and the second analyte. For example, but not by way of limitation, the mass transport limiting membrane overcoating at least one of the sensing layers can include an admixture of a first membrane polymer and a second membrane polymer or a bilayer of the first membrane polymer and the second membrane polymer. A homogeneous membrane can overcoat the sensing layer not overcoated with the admixture or the bilayer, wherein the homogeneous membrane includes only one of the first membrane polymer or the second membrane polymer. Advantageously, the architectures of the analyte sensors disclosed herein readily allow a continuous membrane having a homogenous membrane portion to be disposed upon a first sensing layer and a multi-component membrane portion to be disposed upon a second sensing layer of the analyte sensors, thereby equalizing the permeability values for each analyte concurrently to afford improved sensitivity and detection accuracy. Continuous membrane deposition can take place through sequential dip coating operations in particular embodiments.

In some embodiments, the mass transport limiting membrane can comprise a membrane polymer crosslinked with a crosslinking agent disclosed herein. In some embodiments where there are two mass transport limiting membranes, e.g., a first mass transport limiting membrane and a second mass transport limiting membrane, each membrane can be crosslinked with a different crosslinking agent. For example, but not by way of limitation, the crosslinking agent can result in a membrane that is more restrictive to diffusion of certain compounds, e.g., analytes within the membrane, or less restrictive to diffusion of certain compounds, e.g., by affecting the size of the pores within the membrane. For example, but not by way of limitation, in a sensor that is configured to detect alcohol and glucose, the mass transport limiting membrane overcoating the alcohol-responsive area can have a pore size that restricts the diffusion of compounds larger than alcohol, e.g., glucose, through the membrane.

In some embodiments, where an analyte sensor comprises a multilayered membrane comprising a negatively charged polymer and a mass transport limiting membrane, each membrane can be crosslinked with a different crosslinking agent. In some embodiments, where the analyte sensor comprises a multilayered membrane comprising a negatively charged polymer and a mass transport limiting membrane, each membrane can be crosslinked with the same crosslinking agent.

In some embodiments, crosslinking agents for use in the present disclosure can include polyepoxides, carbodiimide, cyanuric chloride, triglycidyl glycerol, N-hydroxysuccinimide, imidoesters, epichlorohydrin or derivatized variants thereof. In some embodiments, a membrane polymer overcoating one or more sensing layers can be crosslinked with a branched crosslinker, e.g., which can decrease the amount of extractables obtainable from the mass transport limiting membrane. Non-limiting examples of a branched crosslinker include branched glycidyl ether crosslinkers, e.g., including branched glycidyl ether crosslinkers that include two or three or more crosslinkable groups. In some embodiments, the branched crosslinker can include two or more crosslinkable groups, such as polyethylene glycol diglycidyl ether. In some embodiments, the branched crosslinker can include three or more crosslinkable groups, such as polyethylene glycol tetraglycidyl ether. In some embodiments, the membrane polymer can include polyvinylpyridine or a copolymer of vinylpyridine and styrene crosslinked with a branched glycidyl ether crosslinker including two or three crosslinkable groups, such as polyethylene glycol tetraglycidyl ether or polyethylene glycol diglycidyl ether. In some embodiments, the epoxide groups of a polyepoxides, e.g., polyethylene glycol tetraglycidyl ether or polyethylene glycol diglycidyl ether, can form a covalent bond with pyridine or an imidazole via epoxide ring opening resulting in a hydroxyalkyl group bridging a body of the crosslinker to the heterocycle of the membrane polymer.

In some embodiments, the crosslinking agent can be polyethylene glycol diglycidyl ether (PEGDGE). In some embodiments, the PEGDGE used to promote crosslinking (e.g., intermolecular crosslinking) between two or more membrane polymer backbones can exhibit a broad range of suitable molecular weights. In some embodiments, the molecular weight of the PEGDGE can range from about 100 g/mol to about 5,000 g/mol. The number of ethylene glycol repeat units in each arm of the PEGDGE can be the same or different, and can typically vary over a range within a given sample to afford an average molecular weight. In some embodiments, the PEGDGE for use in the present disclosure has an average molecular weight (Mn) from about 200 to 1,000, e.g., about 400. In some embodiments, the crosslinking agent is PEGDGE 400.

In some embodiments, the polyethylene glycol tetraglycidyl ether used to promote crosslinking (e.g., intermolecular crosslinking) between two or more membrane polymer backbones can exhibit a broad range of suitable molecular weights. Up to four polymer backbones can be crosslinked with a single molecule of the polyethylene glycol tetraglycidyl ether crosslinker. The number of ethylene glycol repeat units in each arm of the polyethylene glycol tetraglycidyl ether can be the same or different, and can typically vary over a range within a given sample to afford an average molecular weight.

### 9. Interference Domain

In some embodiments, the sensor of the present disclosure, e.g., implantable portion, can further comprise an interference domain. In some embodiments, the interference domain can include a polymer domain that restricts the flow of one or more interferents, e.g., to the surface of the working electrode. In some embodiments, the interference domain can function as a molecular sieve that allows analytes and other substances that are to be measured by the working electrode to pass through, while preventing passage of other substances such as interferents. In some embodiments, the interferents can affect the signal obtained at the working electrode. Non-limiting examples of interferents include acetaminophen, ascorbate, ascorbic acid, bilirubin, cholesterol, creatinine, dopamine, ephedrine, ibuprofen, L-dopa, methyldopa, salicylate, tetracycline, tolazamide, tolbutamide, triglycerides, urea, and uric acid.

In some embodiments, the interference domain can be located between the working electrode and one or more sensing layers, e.g., alcohol-responsive sensing layer. In some embodiments, non-limiting examples of polymers that can be used in the interference domain include polyurethanes, polymers having pendant ionic groups, and polymers having controlled pore size. In some embodiments, the interference domain is formed from one or more cellulosic derivatives. Non-limiting examples of cellulosic derivatives include polymers such as cellulose acetate, cellulose acetate butyrate, 2-hydroxyethyl cellulose, cellulose acetate phthalate, cellulose acetate propionate, cellulose acetate trimellitate, and the like.

In some embodiments, the interference domain can be part of the mass transport limiting membrane and not a separate membrane.

In some embodiments, the interference domain can include a thin, hydrophobic membrane that is non-swellable and restricts diffusion of high molecular weight species. For example, but not by way of limitation, the interference domain can be permeable to relatively low molecular weight substances, such as hydrogen peroxide, while restricting the passage of higher molecular weight substances, such as ketones, glucose, acetaminophen, and/or ascorbic acid.

In some embodiments, the interference domain can be deposited directly onto the working electrode, e.g., onto the surface of the working electrode. In some embodiments, the interference domain has a thickness, e.g., dry thickness, ranging from about 0.1 μm to about 1,000 μm, e.g., from about 1 μm to about 500 μm, from about 1 μm to about 100 μm, or from about 10 μm to about 100 μm. In some embodiments, the interference domain can have a thickness from about 0.1 μm to about 10 μm, e.g., from about 0.5 μm to about 10 μm, from about 1 μm to about 10 μm, from about 1 μm to about 5 μm, or from about 0.1 μm to about 5 μm. In some embodiments, the sensor can be dipped in the interference domain solution more than once. For example, but not by way of limitation, a sensor (or working electrode) of the present disclosure can be dipped in an interference domain solution at least once, at least twice, at least three times, at least four times, or at least five times to obtain the desired interference domain thickness.

### 10. Manufacturing

The present disclosure further provides methods for manufacturing the presently disclosed analyte sensors that includes one or more sensing layers and one or more working electrodes.

In some embodiments, the method includes depositing a supply of NAD(P), and a NAD(P)-dependent enzyme on a working electrode to provide a sensing layer. In some embodiments, the method can further include adding a multilayered membrane comprising at least one negatively charged polymer on top of the sensing layer.

In some embodiments, the method includes depositing an enzyme composition comprising a supply of NAD(P) and one or more NAD(P)-dependent enzymes, e.g., an NAD(P)-dependent dehydrogenase, on a working electrode. In some embodiments, the enzyme composition can include one or more additional enzymes, e.g., diaphorase, a crosslinking agent, e.g., polyethylene glycol diglycidyl ether, and/or a redox mediator. In some embodiments, the enzyme composition can be deposited onto the surface of a working electrode as one large application which covers the desired portion of the working electrode or in the form of an array of a plurality of enzyme compositions, e.g., spaced apart from each other, to generate one or more sensing layers for detecting one or more analytes. In some embodiments, the method can further include curing the enzyme composition.

In some embodiments, the method can further include adding a multilayered membrane comprising at least one negatively charged polymer on top of the cured enzyme composition.

In some embodiments, the supply of NAD(P) and the NAD(P)-dependent enzyme can be prepared as solutions that dry or cure to solidify after deposition. Therefore, in some embodiments, all layers can be deposited in an automated fashion using small-volume liquid handling or similar techniques for high-throughput sensor fabrication.

In some embodiments, the method can further include adding a membrane composition on top of the cured sensing layer and/or around the entire sensor. In some embodiments, the membrane composition is a multilayered membrane comprising at least one negatively charged polymer, a mass transport limiting membrane, or a combination. In some embodiments, the method can include curing the membrane composition.

### IV. Analyte Monitoring

The present disclosure further provides methods of using the analyte sensors disclosed herein to detect an analyte in vivo. In some embodiments, the present disclosure provides methods for detecting one or more analytes, e.g., one analyte or two analytes. For example, but not by way of limitation, the present disclosure provides methods for detecting one or more analytes including glucose, ketones, lactate, oxygen, hemoglobin A1C, albumin, alcohol, alkaline phosphatase, alanine transaminase, aspartate aminotransferase, bilirubin, blood, urea, nitrogen, calcium, carbon dioxide, chloride, creatinine, hematocrit, lactate, magnesium, oxygen, pH, phosphorus, potassium, sodium, total protein, and/or uric acid using one or more NAD(P)-dependent enzymes. In some embodiments, the analyte can be ketones, alcohol, glucose, and/or lactate using one or more NAD(P)-dependent enzymes. For example, but not by way of limitation, the present disclosure provides methods for detecting one or more ketones. In some embodiments, the present disclosure provides methods for detecting glucose. In some embodiments, the present disclosure provides methods for detecting creatinine. In some embodiments, the present disclosure provides methods for detecting lactate. In some embodiments, the present disclosure provides methods for detecting alcohol.

In some embodiments, the present disclosure provides methods for monitoring in vivo levels of an analyte over time with analyte sensors that include a supply of NAD(P) and one or more NAD(P)-dependent enzymes, e.g., an NAD(P)-dependent dehydrogenase. Generally, monitoring the in vivo concentration of an analyte in a fluid of the body of a subject includes inserting at least partially under a skin surface an in vivo analyte sensor as disclosed herein, contacting the monitored fluid (interstitial, blood, dermal, and the like) with the inserted sensor and generating a sensor signal at the working electrode. The presence and/or concentration of the analyte detected by the analyte sensor can be displayed, stored, forwarded, and/or otherwise processed. A variety of approaches can be employed to determine the concentration of analyte (e.g., glucose, an alcohol, a ketone, and/or lactate) with the disclosed sensors. In some embodiments, monitoring the concentration of analyte using the sensor signal can be performed by coulometric, amperometric, voltammetric, potentiometric, or any other convenient electrochemical detection technique.

In some embodiments, the analyte sensors comprising a multilayered membrane comprising at least one negatively charged polymer display increased stability. In some embodiments, the analyte sensors comprising a multilayered membrane comprising at least one negatively charged polymer exhibit less than a 20% decrease (signal drop) in signal over a period of 12 days. In some embodiments, the analyte sensors comprising a multilayered membrane comprising at least one negatively charged polymer exhibit less than a 20% decrease, less than a 15% decrease, less than a 10% decrease, or less than a 5% decrease in signal over a period of 12 days. In some embodiments, the analyte sensors comprising a multilayered membrane comprising at least one negatively charged polymer exhibit less than a 15% decrease in signal over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days. In some embodiments, the analyte sensors comprising a multilayered membrane comprising at least one negatively charged polymer exhibit less than a 10% decrease in signal over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days. In some embodiments, the analyte sensors comprising a multilayered membrane comprising at least one negatively charged polymer exhibit less than a 20% decrease in signal over a period of 12 days, over a period of 14 days, over a period of 16 days, over a period of 18 days, over a period of 20 days, over a period of 22 days, over a period of 24 days, over a period of 26 days, over a period of 28 days, or over a period of 30 days.

In some embodiments, a method for detecting an analyte includes:
(i) providing an analyte sensor including:
   (a) a first sensing layer disposed upon a surface of a first working electrode, wherein the first sensing layer comprises an NAD(P)-dependent enzyme and a supply of NAD(P);
   (b) a multilayered membrane that overcoats at least a part of the first sensing layer and is permeable to a first analyte, wherein the membrane comprises at least one layer of negatively charged polymer;
(ii) applying a potential to the first working electrode;
(iii) obtaining a first signal at or above an oxidation-reduction potential of the first sensing layer, the first signal being proportional to a concentration of the first analyte in a fluid contacting the first sensing layer; and
(iv) correlating the first signal to the concentration of the first analyte in the fluid.

In some embodiments, a method for detecting one or more alcohols includes:
(i) providing an analyte sensor including:
   (a) an alcohol-responsive sensing layer disposed upon a surface of a first working electrode, wherein the alcohol-responsive sensing layer comprises an NAD(P)-dependent enzyme and a supply of NAD(P);
   (b) a multilayered membrane that overcoats at least a part of the alcohol-responsive sensing layer and is permeable to an analyte, wherein the membrane comprises at least one layer of negatively charged polymer;
(ii) applying a potential to the first working electrode;
(iii) obtaining a first signal at or above an oxidation-reduction potential of the alcohol-responsive sensing layer, the first signal being proportional to a concentration of analyte in a fluid contacting the alcohol-responsive sensing layer; and
(iv) correlating the first signal to the concentration of alcohol in the fluid.

In some embodiments, the method of the present disclosure can further include detecting a second analyte by providing an analyte sensor that includes a second sensing layer and/or exposing an analyte sensor that includes a second sensing layer to a fluid comprising the first analyte and the second analyte. In some embodiments, the analyte sensor for use in a method for detecting a first analyte and a second analyte can further include a second working electrode; and a second sensing layer disposed upon a surface of the second working electrode and responsive to a second analyte differing from the first analyte, where the second sensing layer comprises a second polymer, at least one enzyme responsive to the second analyte covalently bonded to the second polymer and, optionally, a redox mediator covalently bonded to the second polymer; wherein a portion, e.g., second portion, of the mass transport limiting membrane overcoats the second sensing layer. Alternatively, the second sensing layer can be covered by a second mass transport limiting membrane that is separate and/or different than a mass transport limiting membrane that overcoats the first sensing layer. In some embodiments, at least one enzyme responsive to the second analyte comprises an enzyme system comprising multiple enzymes that are collectively responsive to the second analyte.

In some embodiments, the method further includes attaching an electronics unit to the skin of the patient, coupling conductive contacts of the electronics unit to contacts of the sensor, collecting data using the electronics unit regarding a level of analyte from signals generated by the sensor, and forwarding the collected data from electronics unit to a receiver unit, e.g., by RF. In some embodiments, the receiver unit is a mobile telephone. In some embodiments, the mobile telephone includes an application related to the monitored analyte. In some embodiments, analyte information is forwarded by RFID protocol, such as BLUETOOTH®, and the like.

In some embodiments, the analyte sensor can be positioned in a user for automatic analyte sensing, e.g., continuously or periodically. In some embodiments, the level of the analyte can be monitored over a time period ranging from seconds to minutes, hours, days, weeks or months. In some embodiments, the methods disclosed herein can be used to predict future levels of the analyte, based on the obtained information, such as but not limited to current analyte level at time zero, as well as the rate of change of the analyte concentration or amount.

### V. Exemplary Embodiments

(1) In some non-limiting embodiments, the presently disclosed subject matter provides for analyte sensors comprising:
a proximal portion configured to be positioned above a user's skin; and
a distal portion configured to be transcutaneously positioned through the user's skin and in contact with the user's bodily fluid to detect or monitor the analyte in the bodily fluid in vivo; the distal portion comprising:
(i) a first sensing layer disposed upon a surface of a first working electrode, wherein the first sensing layer comprises an NAD(P)-dependent enzyme and a supply of NAD(P); and
(ii) a multilayered membrane that overcoats at least a part of the first sensing layer and is permeable to a first analyte, wherein the membrane comprises at least one layer of negatively charged polymer, and wherein the negatively charged polymer limits transport of the supply of NAD(P) from the first sensing layer.

(2) The analyte sensor of (1), wherein the multilayered membrane comprises between 1 and 3 layers of negatively charged polymer.

(3) The analyte sensor of (1) or (2), wherein the multilayered membrane comprises 1 layer of negatively charged polymer.

(4) The analyte sensor of any one of (1)-(3), wherein the negatively charged polymer comprises negatively charged sulfonate groups.

(5) The analyte sensor of any one of (1)-(4), wherein the negatively charged polymer comprises a copolymer of poly(tetrafluoroethylene) and a poly(perfluorosulfonic acid).

(6) The analyte sensor of (5), wherein the perfluorosulfonic acid has repeat units represented by Formula (I):

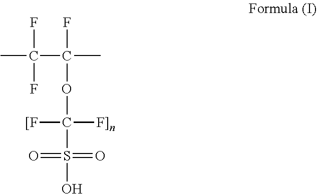

wherein n is an integer from 2 to 4.

(7) The analyte sensor of any one of (1)-(6), wherein the negatively charged polymer comprises AQUIVION® SO₃Li.

(8) The analyte sensor of any one of (1)-(4), wherein the negatively charged polymer comprises a sulfo-phenylated polyphenylene.

(9) The analyte sensor of (8), wherein the negatively charged polymer comprises a sulfo-phenylated polyphenylene polymer having repeat units represented by Formula (II):

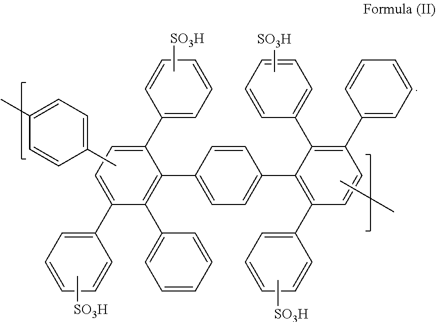

(10) The analyte sensor of any one of (1)-(4), wherein the negatively charged polymer comprises PEMION®.

(11) The analyte sensor of any one of (1)-(4), wherein the negatively charged polymer comprises a copolymer of poly(vinylpyridine) and poly(styrene sulfonate).

(12) The analyte sensor of any one of (1)-(4), wherein the negatively charged polymer comprises poly(4-vinylpyridine-co-4-styrene sulfonic acid salt).

(13) The analyte sensor of any one of (1)-(12), wherein the multilayered membrane further comprises at least one layer of a polymer selected from the group consisting of a polyvinylpyridine, a polyvinylimidazole, a polyacrylate, a polyurethane, a polyether urethane, a polystyrene, a polyacrylamide, and combinations thereof.

(14) The analyte sensor of (13), wherein the multilayered membrane comprises from 1 to 17 layers of a polymer that is not negatively charged.

(15) The analyte sensor of (13) or (14), wherein the multilayered membrane comprises between 1 and 4 layers of poly(4-vinylpyridine).

(16) The analyte sensor of any one of (13)-(15), wherein the multilayered membrane comprises 2 layers of poly(4-vinylpyridine).

(17) The analyte sensor of any one of (1)-(16), wherein the negatively charged polymer limits at least 80% of transport of the supply of NAD(P) from the first sensing layer.

(18) The analyte sensor of any one of (1)-(17), wherein the analyte sensor exhibits less than a 20% decrease in signal over a period of 12 days.

(19) The analyte sensor of any one of (1)-(18), wherein the NAD(P)-dependent enzyme is an NAD(P)-dependent dehydrogenase or a NAD(P)-dependent ketoreductase.

(20) The analyte sensor of any one of (1)-(19), wherein the NAD(P)-dependent enzyme is a glucose dehydrogenase, a lactate dehydrogenase, an alcohol dehydrogenase, a β-hydroxybutyrate dehydrogenase, a phenylalanine hydrogenase, a aldehyde reductase, or a ketoreductase.

(21) The analyte sensor of any one of (1)-(20), wherein the NAD(P)-dependent enzyme is an aldehyde reductase or a ketoreductase.

(22) The analyte sensor of any one of (1)-(21), wherein the analyte is selected from the group consisting of glucose, a ketone, an alcohol, lactate, and combinations thereof.

(23) The analyte sensor of any one of (1)-(22), wherein the analyte is an alcohol.

(24) The analyte sensor of any one of (1)-(23), wherein the first working electrode comprises carbon.

(25) The analyte sensor of any one of (1)-(24), wherein the first sensing layer further comprises diaphorase.

(26) The analyte sensor of any one of (1)-(25), wherein the first sensing layer further comprises a redox mediator.

(27) The analyte sensor of any one of (1)-(26), further comprising: a second sensing layer disposed upon a surface of the first working electrode and responsive to a second analyte differing from the first analyte; wherein the second sensing layer comprises at least one enzyme responsive to the second analyte.

(28) The analyte sensor of any one of (1)-(27), further comprising:
(iii) a second working electrode; and
(iv) a second sensing layer disposed upon a surface of the second working electrode and responsive to a second analyte differing from the first analyte;
wherein the second sensing layer comprises at least one enzyme responsive to the second analyte.

(29) The analyte sensor of (27) or (28), wherein the second sensing layer comprises an NAD(P)-dependent enzyme and a supply of NAD(P).

(30) The analyte sensor of any one of (27)-(29), wherein the multilayered membrane overcoats at least a part of the second sensing layer.

(31) In some non-limiting embodiments, the presently disclosed subject matter provides a method comprising:
(i) applying a potential to a first working electrode of an analyte sensor, wherein the analyte sensor comprises:
   (a) a first sensing layer disposed upon a surface of the first working electrode, wherein the first sensing layer comprises an NAD(P)-dependent enzyme and a supply of NAD(P); and
   (b) a multilayered membrane that overcoats at least a part of the first sensing layer and is permeable to the first analyte, wherein the membrane comprises at least one layer of negatively charged polymer, and wherein the negatively charged polymer limits transport of the supply of NAD(P) from the first sensing layer;
(ii) obtaining a first signal at or above an oxidation-reduction potential of the first sensing layer, the first signal being proportional to a concentration of a first analyte in a fluid contacting the first sensing layer; and
(iii) correlating the first signal to the concentration of the first analyte in the fluid.

### EXAMPLES

The presently disclosed subject matter will be better understood by reference to the following Examples, which are provided as exemplary of the presently disclosed subject matter, and not by way of limitation.

### Example 1: Preparation of Alcohol Sensor with Negatively Charged Poly(Perfluorosulfonic Acid) Membrane

Alcohol was used as a model for this example, but the principle can be applied to many analytes of interest, as described herein. An analyte sensor comprising a poly(4-vinylpyridine) membrane was used as a control.

A poly(4-vinylpyridine) membrane solution was prepared by mixing 4 mL of poly(4-vinylpyridine) at 150 mg/mL in 100 mL of ethanol, 180 μL of polyethylene glycol diglycidyl ether (PEDGDE) 400 at 100 mg/mL in 100% ethanol, and 13.2 μL of polydimethylsiloxane (PDMS) at 100 mg/mL in 100% ethanol. An AQUIVION® membrane solution was prepared by dispersing 4.3 mL AQUIVION® SiO₃Li (Sigma-Aldrich, St. Louis, MO (liquid dispersion, 25% in water)) as a 25% dispersion in deionized water. An osmium-containing poly(4-vinylpyridine)-based polymer membrane solution was prepared by mixing 4 mL of an osmium-containing poly(4-vinylpyridine)-based polymer membrane at 100 mg/mL in a 80:20 ratio (volume by volume) of ethanol:10 mM HEPES (N-2-hydroxyethylpiperazine-N′-2-ethanesulfonic acid) buffer at a pH of 8.0, 1 mL of glycerol triglycidyl ether (Gly3) at 12.5 mg/mL in a 80:20 ratio (volume by volume) of ethanol:HEPES buffer (at a pH of 8.0), and 13.2 μL of PDMS at 100 mg/mL in 100% ethanol.

Analyte sensors for alcohol were washed in deionized water for 1 second and dried for 20 minutes before dipping. All dipping steps were conducted at 55% relative humidity and at a temperature of 21° C. After drying, the analyte sensors were dipped 3 times with a 3 mm/sec (entry and exit) speed into the poly(4-vinylpyridine) membrane solution. The analyte sensors were allowed to dry for 10 minutes after the first dip, 10 minutes after the second dip, and 20 minutes after the third dip. The analyte sensors were then dipped 1 time with a 3 mm/sec (entry and exit) speed into the poly(4-vinylpyridine) membrane solution followed by 20 minutes of drying. Then, the analyte sensors were dipped 4 times with a 3 mm/sec (entry and exit) speed into the AQUIVION® membrane solution. The analyte sensors were allowed to dry for 10 minutes after the first, second, and third dips and for 20 minutes after the fourth dip. The analyte sensors were then dipped twice with a 3 mm/sec (entry and exit) speed into the poly(4-vinylpyridine) membrane solution. The analyte sensors were allowed to dry for 10 minutes after the first dip and 20 minutes after the second dip. Finally, the sensors were dipped twice with a 3 mm/sec (entry and exit) speed into the osmium-containing poly(4-vinylpyridine)-based polymer membrane solution. The analyte sensors were allowed to dry for 10 minutes after the first dip and 20 minutes after the second dip. After dipping was complete, the analyte sensors were stored at 60% relative humidity and at a temperature of 25° C. for 24 hours. After 24 hours, the sensors were transferred to a desiccated vial and aged at 56° C. for 24 hours.

### Example 2: Beaker Stability of the Alcohol Sensor with Negatively Charged Poly(Perfluorosulfonic Acid) Membrane

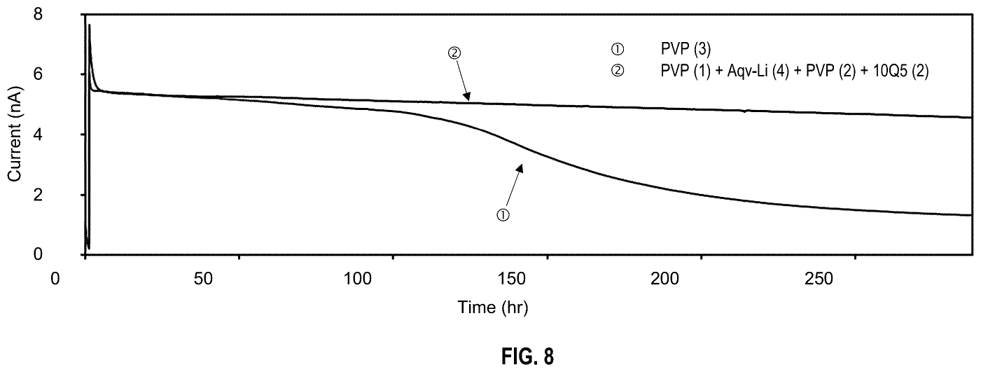

A comparative analyte sensor was prepared with a 3 layer poly(4-vinylpyridine) membrane. The comparative analyte sensor and the sensor prepared with a multilayered membrane comprising a negatively charged polymer were made with the same sensing layer composition that was deposited multiple times on a substrate, which was then cut to form a single sensor. The beaker stability (long-term stability) of the analyte sensor prepared above with a multilayered membrane comprising a negatively charged polymer and the comparative analyte sensor were evaluated by adding 30 mM alcohol into a solution of Dulbecco's phosphate-buffered saline (DPBS) with a pH of 7.4 at stored at 33° C. for 300 hours. Measurements were made using amperometry technique with working electrode poised at positive 40 mV bias to a silver/silver chloride reference electrode in a 3-electrode set-up with a custom potentiostat and a custom control data acquisition software. Temperature was maintained using a jacketed beaker (as a 1 L electrochemical cell) and a circulating water bath. Steady state was maintained through gentle mixing of the cell with a stir plate and magnetic stir bar. The results, shown in FIG. 8, demonstrate that the analyte sensor with a multilayered membrane comprising a negatively charged polymer showed minimal signal drop (decreased stability for detecting alcohol) after 12 days whereas the comparative analyte sensor showed an almost 70% signal drop after 12 days. Thus, the stability of the analyte sensor with a multilayered membrane comprising a negatively charged polymer is significantly better than that of the control analyte sensor.

### Example 3: Preparation of Alcohol Sensor with Negatively Charged Poly(Styrene Sulfonate) Membrane

Alcohol was used as a model for this example, but the principle can be applied to many analytes of interest, as described herein. An analyte sensor comprising a 3 layer poly(4-vinylpyridine) membrane was used as a control.

A poly(4-vinylpyridine) membrane solution was prepared by mixing 4 mL of poly(4-vinylpyridine) at 150 mg/mL in 100 mL of ethanol, 180 μL of PEDGDE 400 at 100 mg/mL in 100% ethanol, and 13.2 μL of PDMS at 100 mg/mL in 100% ethanol. An poly-4-vinylpyridine)-co-poly(styrene sulfonate) membrane solution was prepared by dispersing 4 mL poly(4-vinylpyridine)-co-poly(styrene sulfonate) at 70 mg/mL in deionized water and 200 μL of PEDGDE 200 at 65 mg/mL in deionized water. An osmium-containing poly(4-vinylpyridine)-based polymer membrane solution was prepared by mixing 4 mL of osmium-containing poly(4-vinylpyridine)-based polymer membrane polymer at 100 mg/mL in a 80:20 ratio (volume by volume) of ethanol:10 mM HEPES buffer (at a pH of 8.0), 1 mL of Gly3 at 12.5 mg/mL in a 80:20 ratio (volume by volume) of ethanol:HEPES buffer (at a pH of 8.0), and 13.2 μL of PDMS at 100 mg/mL in 100% ethanol.

Analyte sensors for alcohol were washed in deionized water for 1 second and dried for 20 minutes before dipping. All dipping steps were conducted at 55% relative humidity and at a temperature of 21° C. After drying, the analyte sensors were dipped 3 times with a 3 mm/sec (entry and exit) speed into the poly(4-vinylpyridine) membrane solution. The analyte sensors were allowed to dry for 10 minutes after the first dip, 10 minutes after the second dip, and 20 minutes after the third dip. The analyte sensors were then dipped once with a 3 mm/sec (entry and exit) speed into the poly(4-vinylpyridine) membrane solution followed by 20 minutes of drying. Then, the analyte sensors were dipped 4 times with a 3 mm/sec (entry and exit) speed into the poly(4-vinylpyridine)-co-poly(styrene sulfonate) membrane solution. The analyte sensors were allowed to dry for 10 minutes after the first, second, and third dips and for 20 minutes after the fourth dip. The analyte sensors were then dipped twice with a 1 mm/sec (entry and exit) speed into the poly(4-vinylpyridine) membrane solution. The analyte sensors were allowed to dry for 10 minutes after the first dip and 20 minutes after the second. Finally, the sensors were dipped twice with a 1 mm/sec (entry and exit) speed into the osmium-containing poly(4-vinylpyridine)-based polymer membrane solution. The analyte sensors were allowed to dry for 10 minutes after the first dip and 20 minutes after the second dip. After dipping was complete, the sensors were stored at 60% relative humidity and at a temperature of 25° C. for 24 hours. After 24 hours, the sensors were transferred to a desiccated vial and aged at 56° C. for 24 hours.

### Example 4: Beaker Stability of Alcohol Sensor with Negatively Charged Poly(Styrene Sulfonate) Membrane

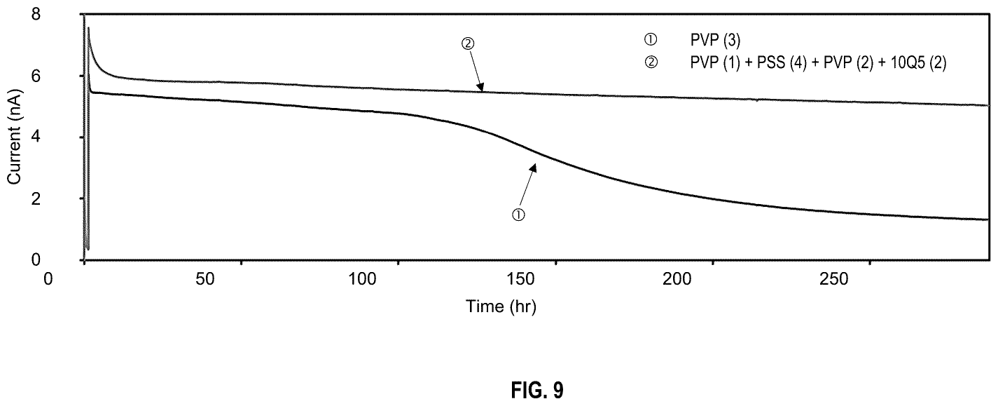

A comparative analyte sensor was prepared with a 3 layer poly(4-vinylpyridine) membrane. Both sensors were made with the same sensing layer composition that was deposited multiple times on a substrate, which was then cut to form a single sensor. The analyte sensor prepared above with a multilayered membrane with a negatively charged polymer and the comparative analyte sensor were evaluated by adding 30 mM alcohol into DPBS buffer with a pH of 7.4 and stored at 33° C. for 300 hours. Measurements were made using amperometry technique with working electrode poised at positive 40 mV bias to a silver/silver chloride reference electrode in a 3-electrode set-up with a custom potentiostat and a custom control/data acquisition software. Temperature was maintained using a jacketed beaker (as a 1 L electrochemical cell) and a circulating water bath. Steady state was maintained through gentle mixing of the cell with a stir plate and magnetic stir bar. As shown in FIG. 9, the analyte sensor with a multilayered membrane comprising a negatively charged polymer showed minimal signal drop over 12 days whereas the comparative analyte sensor showed at least a 70% signal drop over 12 days. Thus, the stability of the analyte sensor with a multilayered membrane comprising a negatively charged polymer is significantly better than that of the control analyte sensor.

### Example 5: Preparation of a 5 wt % Solution of PEMION® Ionomer in Solvent

To a 20 mL glass scintillation vial with a stir bar was added 9.5 g of suitable alcohol or solvent. The vial was placed on a magnetic stirring hot plate and stirred at 300 rpm. 0.5 grams of PEMION® ionomer (Ionomr Innovations Inc., Vancouver, BC, Canada) was slowly added to the solvent over the course of 10 seconds. The resulting 5 wt % polymer suspension was stirred at 300 rpm with heating at 60° C. for 48 hours, giving a viscous and uniform polymer solution. Slow addition of the ionomer to the solvent helped prevent clumping and improved dissolution characteristics. After cooling to room temperature, the solution was filtered through a 55 mm diameter 11 μm pore size Grade 1 Whatman filter paper to remove any insoluble materials before further use. Additional solutions of PEMION® of Examples 7-9 were prepared by varying the solvent and the amount of PEMION® added.

### Example 6: Ethanol Sensors Comprising PEMION® Membrane Layer

The membrane dipping solutions of Examples 7-9 were generated with PEMION® Mid-High IEC Proton Exchange Ionomer (P/N PP1-HNN8-00-X (Ionomr Innovations Inc., Vancouver, BC, Canada)) and used to create ethanol analyte sensors. PEMION® membrane dipping solutions were made by stirring at 65° C. The solvent ratios given are in mass (not volume). Additional membrane dipping solutions of Examples 7-9 were prepared as follows: A poly(4-vinylpyridine) membrane solution was prepared by mixing 4 mL of poly(4-vinylpyridine) at 150 mg/mL in 100 mL of ethanol, 180 μL of PEDGDE 400 at 100 mg/mL in 100% ethanol, and 13.2 μL of PDMS at 100 mg/mL in 100% ethanol. An osmium-containing poly(4-vinylpyridine)-based polymer membrane solution was prepared by mixing 4 mL of osmium-containing poly(4-vinylpyridine)-based polymer membrane polymer at 100 mg/mL in a 80:20 ratio (volume by volume) of ethanol:10 mM HEPES buffer (at a pH of 8.0), 1 mL of Gly3 at 12.5 mg/mL in a 80:20 ratio (volume by volume) of ethanol:HEPES buffer (at a pH of 8.0), and 13.2 μL of PDMS at 100 mg/mL in 100% ethanol.

### Example 7: Ethanol Sensor Prepared with 3 wt % PEMION® in Ethanol Membrane Solution

To prepare analyte sensors using a membrane solution with 3 wt % PEMION® in ethanol: Analyte sensors for alcohol were washed with deionized water once with a 5 mm/sec enter speed, a 1 second dwell time, and a 5 mm/sec exit speed and subsequently dried for 20 minutes before dipping. All dipping steps were conducted between 45% and 50% relative humidity and the temperature was not controlled. After drying, the analyte sensors were dipped once into a poly(4-vinylpyridine) membrane solution with a 2 mm/sec entry, a 1 second dwell time, and an 8 mm/sec exit speed. The analyte sensors were allowed to dry for 20 minutes. The analyte sensors were then dipped once with a 2 mm/sec entry speed, a 5 second dwell time, and a 3 mm/sec exit speed into the 3 wt % PEMION with a pH˜6 (LiOH) in EtOH membrane solution followed by 20 minutes of drying. The analyte sensors were then dipped twice into a poly(4-vinylpyridine) membrane solution with the first dip having a 2 mm/sec entry, a 5 second dwell time, and a 8 mm/sec exit speed. After allowing the analyte sensors to dry for 10 minutes, the second dip had a 2 mm/sec entry time, a 1 second dwell time, and a 6 mm/sec exit speed. The analyte sensors were allowed to dry for 20 minutes after the second dip. Finally, the sensors were dipped twice with a 3 mm/sec entry speed, a 1 second dwell time, and a 10 mm/sec exit speed into an osmium-containing poly(4-vinylpyridine)-based polymer membrane solution. The analyte sensors were allowed to dry for 10 minutes after the first dip and 20 minutes after the second dip. After dipping was complete, the analyte sensors were stored at 60% relative humidity and at a temperature of 25° C. for 24 hours. After 24 hours, the sensors were transferred to a desiccated vial and aged at 56° C. for 24 hours.

### Example 8: Ethanol Sensor Prepared with 3 wt % PEMION® in 1:1 H₂O:Isopropanol Membrane Solution

To prepare analyte sensors using a membrane solution of 3 wt % PEMION® in 1:1 H₂O:isopropanol: Analyte sensors for alcohol were washed once in deionized water with a 5 mm/sec entry speed, a 1 second dwell time, and a 5 mm/sec exit speed in deionized water and dried for 20 minutes before dipping. All dipping steps were conducted between 45% and 50% relative humidity and the temperature was not controlled. After drying, the analyte sensors were dipped once with a 2 mm/sec entry speed, a 1 second dwell time, and a 8 mm/sec exit speed into the poly(4-vinylpyridine) membrane solution. The analyte sensors were allowed to dry for 20 minutes. The analyte sensors were then dipped once with a 2 mm/sec entry speed, a 5 second dwell time, and a 3 mm/sec exit speed into the 3 wt % PEMION with a pH˜10 (LiOH) in 1:1 H₂O:isopropanol membrane solution followed by 20 minutes of drying. The analyte sensors were then dipped twice into a poly(4-vinylpyridine) membrane solution with the first dip having a 2 mm/sec entry speed, a 5 second dwell time, and a 8 mm/sec exit speed. After allowing the analyte sensors to dry for 10 minutes, the second dip had a 2 mm/sec entry speed, a 1 second dwell time, and a 6 mm/sec exit speed. The analyte sensors were allowed to dry for 20 minutes after the second dip. Finally, the sensors were dipped twice with a 3 mm/sec entry speed, a 1 second dwell time, and a 10 mm/sec exit speed into an osmium-containing poly(4-vinylpyridine)-based polymer membrane solution. The analyte sensors were allowed to dry for 10 minutes after the first dip and 20 minutes after the second dip. After dipping was complete, the analyte sensors were stored at 60% relative humidity and at a temperature of 25° C. for 24 hours. After 24 hours, the sensors were transferred to a desiccated vial and aged at 56° C. for 24 hours.

### Example 9: Ethanol Sensor Prepared with 1 wt % PEMION® in Ethanol Membrane Solution

To prepare analyte sensors using a membrane solution with 1 wt % PEMION® in EtOH: Analyte sensors for alcohol were washed once with a 5 mm/sec entry speed, a 1 second dwell time, and a 5 mm/sec exit speed in deionized water and dried for 20 minutes before dipping. All dipping steps were conducted between 45% and 50% relative humidity and the temperature was not controlled. After drying, the analyte sensors were dipped 3 times each with a 2 mm/sec entry speed, a 1 second dwell time, and a 8 mm/sec exit speed and a 10 minute drying time between dips into the poly(4-vinylpyridine) membrane solution. The analyte sensors were allowed to dry for 20 minutes. The analyte sensors were then dipped 4 times each with a 2 mm/sec entry speed, a 5 second dwell time, and a 0.5 mm/sec exit speed with a 10 minute drying time between dips into the 1 wt % PEMION® with a pH=5 (no LiOH) in EtOH membrane solution followed by 20 minutes of drying. The analyte sensors were then dipped twice into a poly(4-vinylpyridine) membrane solution with the 1st dip having a 2 mm/sec entry speed, a 5 second dwell time, and a 8 mm/sec exit speed. After allowing the analyte sensors to dry for 10 minutes, the second dip had a 2 mm/sec entry speed, a 1 second dwell time, and a 6 mm/sec exit speed. The analyte sensors were allowed to dry for 20 minutes after the second dip. Finally, the sensors were dipped twice with a 2 mm/sec entry speed, a 1 second dwell time, and a 10 mm/sec exit speed into an osmium-containing poly(4-vinylpyridine)-based polymer membrane solution. The analyte sensors were allowed to dry for 10 minutes after the first dip and 20 minutes after the second dip. After dipping was complete, the analyte sensors were stored at 60% relative humidity and at a temperature of 25° C. for 24 hours. After 24 hours, the sensors were transferred to a desiccated vial and aged at 56° C. for 24 hours.

### Example 10: Beaker Testing of Analyte Sensor Comprising PEMION® Membrane Layer

The analyte sensors prepared above with a multilayered membrane with PEMION® negatively charged polymers were evaluated at 33° C. by adding 30 mM ethanol (200 proof) into 10 mM DPBS buffer with a pH of 7.4 which was diluted 10 times with deionized water. Waited 15 minutes after each addition before evaluation. Beaker tests were conducted using amperometry.

Measurements were made using amperometry technique with working electrode poised at positive 40 mV bias to a silver/silver chloride reference electrode in a 3-electrode set-up with a custom potentiostat and a custom control/data acquisition software. Temperature was maintained using a jacketed beaker (as a 1 L electrochemical cell) and a circulating water bath. Steady state was maintained through gentle mixing of the cell with a stir plate and magnetic stir bar.

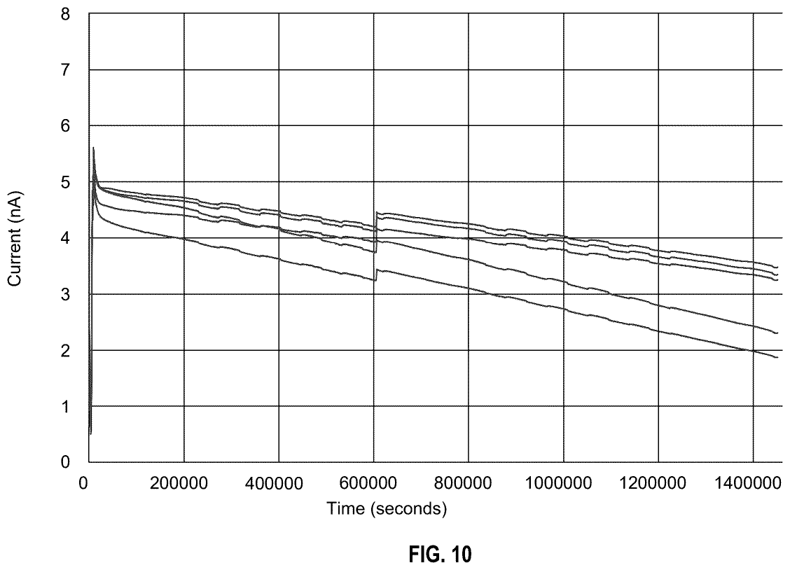

Stability testing at 30 mM ethanol was used to compare with and without the PEMION® membrane. Under specific solvent and dipping conditions, PEMION® layers could be generated within the membrane which improved performance (higher stability and consistency). As shown in FIG. 10, analyte sensors comprising a multilayered membrane comprising PEMION® showed good stability and consistency.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification. As one of ordinary skill in the art will readily appreciate from the disclosed subject matter of the presently disclosed subject matter, processes, machines, manufacture, compositions of matter, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

1. An analyte sensor comprising:
a proximal portion configured to be positioned above a user's skin; and
a distal portion configured to be transcutaneously positioned through the user's skin and in contact with the user's bodily fluid to detect or monitor the analyte in the bodily fluid in vivo; the distal portion comprising:
(i) a first sensing layer disposed upon a surface of a first working electrode, wherein the first sensing layer comprises an NAD(P)-dependent enzyme and a supply of NAD(P); and
(ii) a multilayered membrane that overcoats at least a part of the first sensing layer and is permeable to a first analyte, wherein the membrane comprises at least one layer of negatively charged polymer, and wherein the negatively charged polymer limits transport of the supply of NAD(P) from the first sensing layer.

2. The analyte sensor of claim 1, wherein the multilayered membrane comprises from 1 to 3 layers of negatively charged polymer.

3. (canceled)

4. The analyte sensor of claim 1, wherein the negatively charged polymer comprises negatively charged sulfonate groups.

5. The analyte sensor of claim 1, wherein the negatively charged polymer comprises a copolymer of poly(tetrafluoroethylene) and a poly(perfluorosulfonic acid).

6. The analyte sensor of claim 5, wherein the perfluorosulfonic acid has repeat units represented by Formula (I): 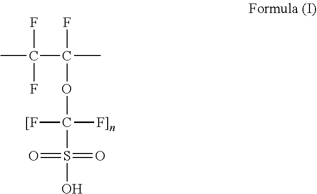
wherein n is an integer from 2 to 4.

7. (canceled)

8. The analyte sensor of claim 1, wherein the negatively charged polymer comprises a sulfo-phenylated polyphenylene.

9. The analyte sensor of claim 8, wherein the negatively charged polymer comprises a sulfo-phenylated polyphenylene polymer having repeat units represented by Formula (II):

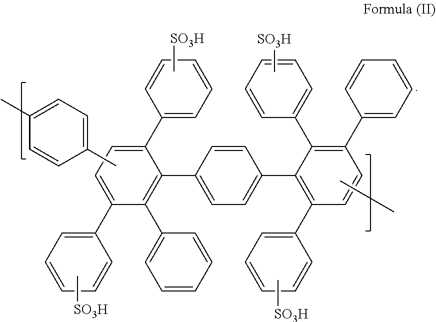

10. (canceled)

11. The analyte sensor of claim 1, wherein the negatively charged polymer comprises a copolymer of poly(vinylpyridine) and poly(styrene sulfonate).

12. The analyte sensor of claim 1, wherein the negatively charged polymer comprises poly(4-vinylpyridine-co-4-styrene sulfonic acid salt).

13. The analyte sensor of claim 1, wherein the multilayered membrane further comprises at least one layer of a polymer selected from the group consisting of a polyvinylpyridine, a polyvinylimidazole, a polyacrylate, a polyurethane, a polyether urethane, a polystyrene, a polyacrylamide, and combinations thereof.

14. The analyte sensor of claim 13, wherein the multilayered membrane comprises from 1 to 5 layers of a polymer that is not negatively charged.

15. (canceled)

16. (canceled)

17. The analyte sensor of claim 1, wherein the negatively charged polymer limits transport of at least 80% of the supply of NAD(P) from the first sensing layer over a period of 12 days.

18. The analyte sensor of claim 1, wherein the analyte sensor exhibits less than a 20% decrease in signal over a period of 12 days.

19. The analyte sensor of claim 1, wherein the NAD(P)-dependent enzyme is an NAD(P)-dependent dehydrogenase or an NAD(P)-dependent ketoreductase.

20. The analyte sensor of claim 1, wherein the NAD(P)-dependent enzyme is a glucose dehydrogenase, a lactate dehydrogenase, an alcohol dehydrogenase, a β-hydroxybutyrate dehydrogenase, a phenylalanine dehydrogenase, an aldehyde reductase, or a ketoreductase.

21. The analyte sensor of claim 1, wherein the NAD(P)-dependent enzyme is an aldehyde reductase or a ketoreductase.

22. The analyte sensor of claim 1, wherein the analyte is selected from the group consisting of glucose, a ketone, an alcohol, lactate, and combinations thereof.

23. The analyte sensor of claim 1, wherein the first analyte is an alcohol.

24. (canceled)

25. The analyte sensor of claim 1, wherein the first sensing layer further comprises diaphorase.

26. The analyte sensor of claim 1, wherein the first sensing layer further comprises a redox mediator.

27. (canceled)

28. The analyte sensor of claim 1, further comprising:
a second working electrode; and
a second sensing layer disposed upon a surface of the second working electrode and responsive to a second analyte differing from the first analyte;
wherein the second sensing layer comprises at least one enzyme responsive to the second analyte.

29. The analyte sensor of claim 28, wherein the second sensing layer comprises an NAD(P)-dependent enzyme and a supply of NAD(P).

30. The analyte sensor of claim 28, wherein the multilayered membrane overcoats at least a part of the second sensing layer.

31. A method comprising:
(i) applying a potential to a first working electrode of an analyte sensor, wherein the analyte sensor comprises:
a proximal portion configured to be positioned above a user's skin; and
a distal portion configured to be transcutaneously positioned through the user's skin and in contact with the user's bodily fluid to detect or monitor the analyte in the bodily fluid in vivo; the distal portion comprising:
(a) a first sensing layer disposed upon a surface of the first working electrode, wherein the first sensing layer comprises an NAD(P)-dependent enzyme and a supply of NAD(P); and
(b) a multilayered membrane that overcoats at least a part of the first sensing layer and is permeable to a first analyte, wherein the membrane comprises at least one layer of negatively charged polymer, wherein the negatively charged polymer limits transport of the supply of NAD(P) from the first sensing layer;
(ii) obtaining a first signal at or above an oxidation-reduction potential of the first sensing layer, the first signal being proportional to a concentration of the first analyte in a fluid contacting the first sensing layer; and
(iii) correlating the first signal to the concentration of the first analyte in the fluid.
